(19) United States
(12) Patent Application Publication
(10) Pub. No.: US 2022/0056053 A1
(43) Pub. Date: Feb. 24, 2022

(54) SYNTHESIS OF CRAC CHANNEL INHIBITORS

(71) Applicant: CalciMedica, Inc., La Jolla, CA (US)

(72) Inventors: Kenneth A. STAUDERMAN, San Diego, CA (US); Michael DUNN, La Jolla, CA (US); Jeffrey P. WHITTEN, Santee, CA (US); Evan ROGERS, San Diego, CA (US)

(21) Appl. No.: 17/519,239

(22) Filed: Nov. 4, 2021

Related U.S. Application Data
(63) Continuation of application No. PCT/US2020/031506, filed on May 5, 2020.
(60) Provisional application No. 62/843,822, filed on May 6, 2019.

(51) Int. Cl. C07F 5/02; C07D 405/04; C07C 45/63

(52) U.S. Cl. CPC C07F 5/025; C07C 45/63; C07D 405/04

(57) ABSTRACT

A convergent synthetic method for the production of CRAC channel inhibitors is described herein. The synthetic method provides a method for producing highly pure CRAC channel inhibitors for clinical testing.

### CROSS-REFERENCE

This application is a continuation of International Application No. PCT/US2020%031506, filed May 5, 2020, which claims the benefit of U.S. Provisional Application No. 62/843,822, filed May 6, 2019, which is hereby incorporated by reference in its entirety herein.

### BACKGROUND

Calcium plays a vital role in cell function and survival. For example, calcium is a key element in the transduction of signals into and within cells. Cellular responses to growth factors, neurotransmitters, hormones and a variety of other signal molecules are initiated through calcium-dependent processes.

Virtually all cell types depend in some manner upon the generation of cytoplasmic Ca²⁺ signals to regulate cell function, or to trigger specific responses. Cytosolic Ca²⁺ signals control a wide array of cellular functions ranging from short-term responses such as contraction and secretion to longer-term regulation of cell growth and proliferation. Usually, these signals involve some combination of release of Ca²⁺ from intracellular stores, such as the endoplasmic reticulum (ER), and influx of Ca²⁺ across the plasma membrane. In one example, cell activation begins with an agonist binding to a surface membrane receptor, which is coupled to phospholipase C (PLC) through a G-protein mechanism. PLC activation leads to the production of inositol 1,4,5-triphosphate (IP3), which in turn activates the IP3 receptor causing release of Ca²⁺ from the ER. The fall in ER Ca²⁺ then signals to activate plasma membrane store-operated calcium (SOC) channels.

Store-operated calcium (SOC) influx is a process in cellular physiology that controls such diverse functions such as, but not limited to, refilling of intracellular Ca²⁺ stores (Putney et al. Cell, 75, 199-201, 1993), activation of enzymatic activity (Fagan et al., J. Biol. Chem. 275:26530-26537.2000), gene transcription (Lewis, Annu. Rev. Immunol. 19:497-521.2001), cell proliferation (Nunez et al., J. Physiol. 571.1, 57-73, 2006), and release of cytokines (Winslow et al., Curr. Opin. Immunol. 15:299-307,2003). In some nonexcitable cells, e.g., blood cells, immune cells, hematopoietic cells, T lymphocytes and mast cells. SOC influx occurs through calcium release-activated calcium (CRAC) channels, a type of SOC channel.

The calcium influx mechanism has been referred to as store-operated calcium entry (SOCE). Stromal interaction molecule (STIM) proteins are an essential component of SOC channel function, serving as the sensors for detecting the depletion of calcium from intracellular stores and for activating SOC channels.

### SUMMARY

One aspect described herein is a process for the synthesis of compounds of Formula (I):

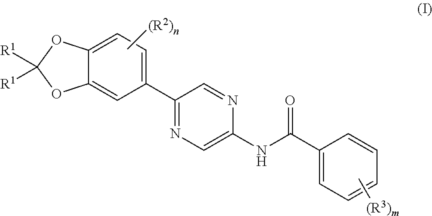

or pharmaceutically acceptable salts thereof, wherein:
R¹ is independently selected at each occurrence from hydrogen, halogen and C₁-C₃ alkyl optionally substituted with one or more substituents independently selected at each occurrence from halogen, —OR′, —CN, —N(R′)₂ and —NO₂.
R² and R³ are independently selected at each occurrence from halogen and C₁-C₃ alkyl optionally substituted with one or more substituents independently selected at each occurrence from halogen, —OR′, —CN, —N(R′)₂ and —NO₂:
or, when both R¹ are independently C₁-C₃ alkyl, the two R¹ groups are taken together with the atom to which they are attached to form a carbocycle;
n is 0, 1, 2 or 3;
m is 0, 1, 2, 3, 4, or 5; and
R′ is independently selected at each occurrence from hydrogen; and C₁₋₄ alkyl. C₂₋₄ alkenyl, and C₂₋₄ alkynyl, each optionally substituted with one or more substituents independently selected at each occurrence from halogen, —CN, —NO₂, —OH, —NH₂, and OCH₃:
   wherein the process comprises contacting a compound of Formula (I-A)

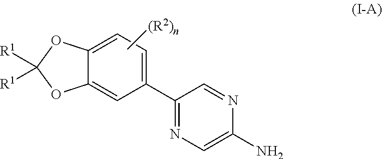

with a compound of Formula (I-B)

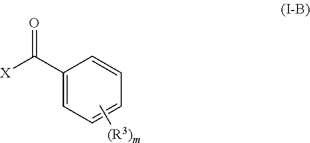

in the presence of a tertiary amine base and an aprotic polar solvent, wherein X is —Cl, —Br, —I, —CN, —N₃, —OCH₃, —OCH₂CH₃, —OC₆H₅, —OC₆H₄-4-NO₂, —OC(O)CH₃, —OC(O)C₆H₅, —O(SO₂)CH₃, or —O(SO₂)C₆H₄-4-CH₃.

In some embodiments, the tertiary amine base is selected from the group consisting of pyridine, triethylamine, triisopropyl amine, 2-tert-butyl-1,1,3,3-tetramethylguanidine, 4-dimethylaminopyridine. N,N-diisopropylethylamine and N-methylmorpholine.

In some embodiments, the aprotic polar solvent is selected from the group consisting of chloroform, dichloromethane, and mixtures thereof.

In some embodiments, the compound of Formula (I-A)

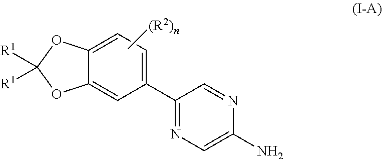

is synthesized by treating a compound of formula (I-C)

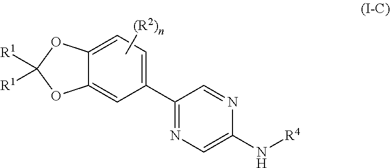

with an acid, wherein R⁴ is selected from the group consisting of trityl, t-butyl, t-butoxycarbonyl, p-tolyl, benzoyl, acetyl and benzyl.

In some embodiments, the acid is selected from the group consisting of trifluoroacetic acid, sulfuric acid and hydrochloric acid.

In some embodiments, the compound of Formula (I-A)

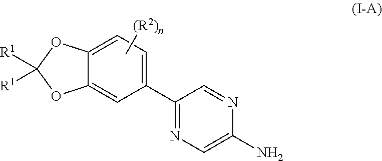

is synthesized by subjecting a compound of formula (I-C)

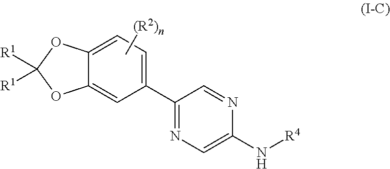

to a hydrogenation, wherein R⁴ is selected from the group consisting of trityl, t-butyl, p-tolyl, and benzyl.

In some embodiments, the compound of formula (I-C)

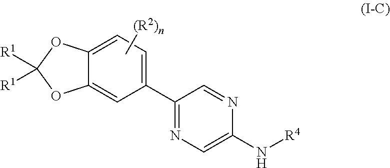

is synthesized by coupling a compound of formula (I-D)

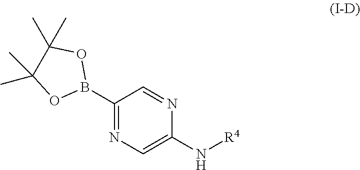

and a compound of formula (I-E)

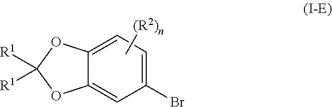

in the presence of a coupling catalyst.

In some embodiments, the coupling catalyst is a palladium-based catalyst. In some embodiments, the palladium-based catalyst is selected from the group consisting of Pd(PPh₃)₄, Pd(dppf)Cl₂ and PdCl₂(PPh₃)₄.

In some embodiments, the coupling is conducted at a temperature from about 80° C. to about 90° C.

In some embodiments, the compound of formula (I-D)

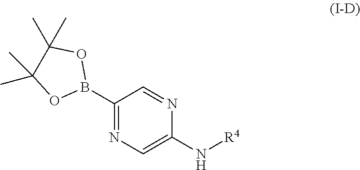

is synthesized by treating a compound of formula (I-F)

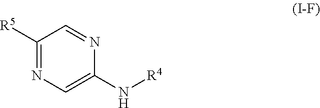

with bis(pinacolato)diboron in the presence of a second palladium-based catalyst, a base and a polar solvent, wherein R⁵ is independently selected from a halogen, —O(SO₂)C₆H₄-4-CH₃, and —O(SO₂)CH₃.

In some embodiments, the second palladium-based catalyst is Pd(dppf)Cl₂.

In some embodiments, the base is potassium acetate.

In some embodiments, the compound of formula (I-F) is

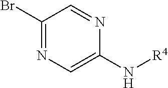

and is synthesized from 2-amino-5-bromopyrazine.

In some embodiments, the compound of formula (I-F) is a crystalline solid.

In some embodiments, the compound of formula (I-B)

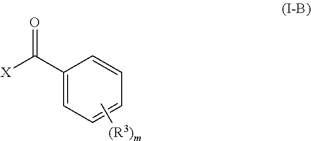

is synthesized by treating a compound of formula (I-G)

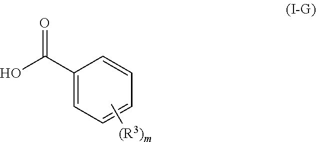

with an acyl halide preparation agent.

In some embodiments, the acyl halide preparation agent is selected from the group consisting of oxalyl chloride, thionyl chloride, phosphoryl chloride, and phosphorus trichloride.

In some embodiments, R¹ is independently selected at each occurrence from hydrogen, halogen and C₁-C₃ alkyl optionally substituted with one or more substituents independently selected at each occurrence from halogen, —OH, —OCH₃, —CN, —NH₂, and —NO₂; and R² and R³ are independently selected at each occurrence from halogen and C₁-C₃ alkyl optionally substituted with one or more substituents independently selected at each occurrence from halogen, —OH, —OCH₃, —CN, —NH₂, and —NO₂.

One aspect described herein is a process for the synthesis of a compound of Formula (II):

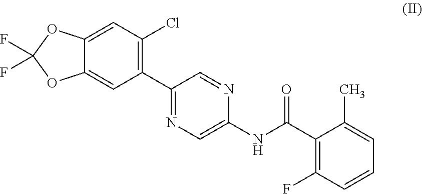 [SMILES: Cc1cccc(F)c1C(=O)Nc1cnc(-c2cc3c(cc2Cl)OC(F)(F)O3)cn1]

or a pharmaceutically acceptable salt thereof, wherein the process comprises contacting a compound of Formula (II-A)

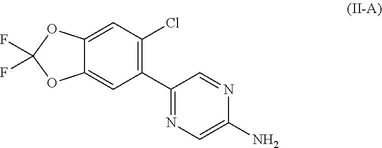 [SMILES: Nc1cnc(-c2cc3c(cc2Cl)OC(F)(F)O3)cn1]

with a compound of Formula (II-B)

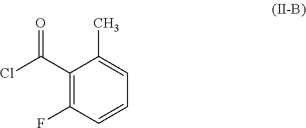

in the presence of a tertiary amine base and an aprotic polar solvent.

In some embodiments, the tertiary amine base is selected from the group consisting of pyridine, triethylamine, triisopropyl amine, 2-tert-butyl-1,1,3,3-tetramethylguanidine, 4-dimethylaminopyridine, N,N-diisopropylethylamine and N-methylmorpholine.

In some embodiments, the aprotic polar solvent is selected from the group consisting of chloroform, dichloromethane, and mixtures thereof.

In some embodiments, the compound of Formula (II-A)

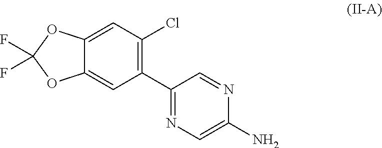 [SMILES: Nc1cnc(-c2cc3c(cc2Cl)OC(F)(F)O3)cn1]

is synthesized by treating a compound of formula (II-C)

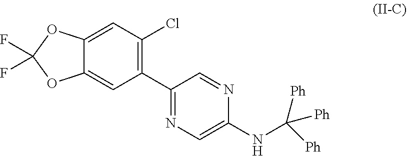 [SMILES: FC1(F)Oc2cc(Cl)c(-c3cnc(NC(c4ccccc4)(c4ccccc4)c4ccccc4)cn3)cc2O1]

with an acid.

In some embodiments, the acid is selected from the group consisting of trifluoroacetic acid, sulfuric acid and hydrochloric acid.

In some embodiments, the compound of Formula (II-A)

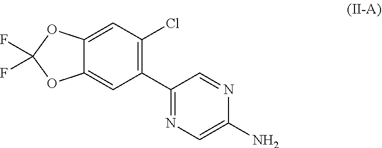 [SMILES: Nc1cnc(-c2cc3c(cc2Cl)OC(F)(F)O3)cn1]

is synthesized by subjecting a compound of formula (II-C)

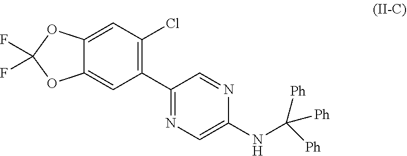 [SMILES: FC1(F)Oc2cc(Cl)c(-c3cnc(NC(c4ccccc4)(c4ccccc4)c4ccccc4)cn3)cc2O1]

to a hydrogenation.

In some embodiments, the compound of formula (II-C)

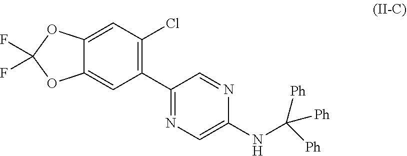 [SMILES: FC1(F)Oc2cc(Cl)c(-c3cnc(NC(c4ccccc4)(c4ccccc4)c4ccccc4)cn3)cc2O1]

is synthesized by coupling a compound of formula (II-D)

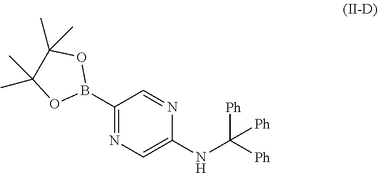 [SMILES: CC1(C)OB(c2cnc(NC(c3ccccc3)(c3ccccc3)c3ccccc3)cn2)OC1(C)C]

and a compound of formula (II-E)

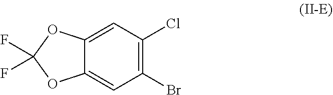 [SMILES: FC1(F)Oc2cc(Cl)c(Br)cc2O1]

in the presence of a coupling catalyst.

In some embodiments, the coupling catalyst is a palladium-based catalyst.

In some embodiments, the palladium-based catalyst is selected from the group consisting of Pd(PPh₃)₄, Pd(dppf)Cl₂ and PdCl₂(PPh₃)₄.

In some embodiments, the coupling is conducted at a temperature from about 80° C. to about 90° C.

In some embodiments, the compound of formula (II-D)

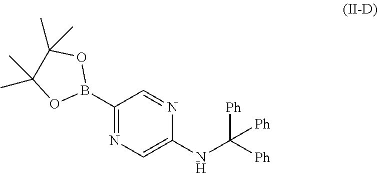 [SMILES: CC1(C)OB(c2cnc(NC(c3ccccc3)(c3ccccc3)c3ccccc3)cn2)OC1(C)C]

is synthesized by treating a compound of formula (II-F)

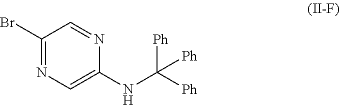 [SMILES: Brc1cnc(NC(c2ccccc2)(c2ccccc2)c2ccccc2)cn1]

with bis(pinacolato)diboron in the presence of a second palladium-based catalyst, a base and a polar solvent.

In some embodiments, the second palladium-based catalyst is Pd(dppf)Cl₂.

In some embodiments, the base is potassium acetate.

In some embodiments, the polar solvent is 1,4-dioxane.

In some embodiments, the compound of formula (II-F)

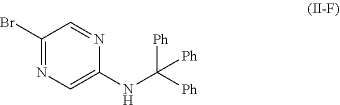 [SMILES: Brc1cnc(NC(c2ccccc2)(c2ccccc2)c2ccccc2)cn1]

is synthesized from 2-amino-5-bromopyrazine.

In some embodiments, the compound of formula (II-F) is a crystalline solid.

In some embodiments, the compound of formula (II-B)

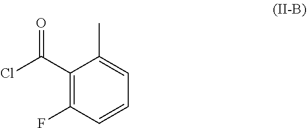

is formed by treating a compound of formula (II-G)

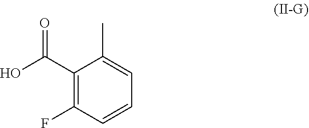

with an acyl halide preparation agent.

In some embodiments, the acyl halide preparation agent is selected from the group consisting of oxalyl chloride, thionyl chloride, phosphoryl chloride, and phosphorus trichloride.

### INCORPORATION BY REFERENCE

All publications, patents, and patent applications mentioned in this specification are herein incorporated by reference to the same extent as if each individual publication, patent, or patent application was specifically and individually named as incorporated by reference.

### DETAILED DESCRIPTION

### Definitions

Unless defined otherwise, all technical and scientific terms used herein have the same meaning as is commonly understood in the field to which the claimed subject matter belongs. In the event that there is a plurality of definitions for terms herein, those in this section prevail. All patents, patent applications, publications and published nucleotide and amino acid sequences (e.g., sequences available in GenBank or other databases) referred to herein are incorporated by reference. Where reference is made to a URL or other such identifier or address, it is understood that such identifiers can change and particular information on the internet can come and go, but equivalent information is found by searching the internet. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of“or” means “and/or” unless stated otherwise.

Furthermore, use of the term “including” as well as other forms, such as “include”, “includes,” and “included,” is not limiting.

The section headings used herein are for organizational purposes only and are not to be construed as limiting the subject matter described.

Definition of standard chemistry terms are found in reference works, including but not limited to, Carey and Sundberg “ADVANCED ORGANIC CHEMISTRY 4TH ED.” Vols. A (2000) and B (2001), Plenum Press, New York. Unless otherwise indicated, conventional methods of mass spectroscopy, NMR, HPLC, protein chemistry, biochemistry, recombinant DNA techniques and pharmacology, within the skill of the art are employed.

The term “CRAC channel inhibitor” refers to inhibitors that suppress calcium release activated channel (CRAC), which are specialized plasma membrane Ca²⁺ ion channels that slowly replenish depleted levels of calcium in the endoplasmic reticulum.

The terms “inhibits”, “inhibiting”, or “inhibitor” of CRAC channel activity, as used herein, refer to inhibition of store operated calcium channel activity or calcium release activated calcium channel activity.

As used herein, C₁-Cₓ includes C₁-C₂, C₁-C₃ . . . C₁-Cₓ, C₁-Cₓ refers to the number of carbon atoms that make up the moiety to which it designates (excluding optional substituents).

An “alkyl” group refers to an aliphatic hydrocarbon group. The alkyl groups may or may not include units of unsaturation. The alkyl moiety may be a “saturated alkyl” group, which means that it does not contain any units of unsaturation (i.e. a carbon-carbon double bond or a carbon-carbon triple bond). The alkyl group may also be an “unsaturated alkyl” moiety, which means that it contains at least one unit of unsaturation. The alkyl moiety, whether saturated or unsaturated, may be branched, straight chain, or cyclic.

The “alkyl” group may have 1 to 6 carbon atoms (whenever it appears herein, a numerical range such as “1 to 6” refers to each integer in the given range; e.g., “1 to 6 carbon atoms” means that the alkyl group may consist of 1 carbon atom, 2 carbon atoms, 3 carbon atoms, etc., up to and including 6 carbon atoms, although the present definition also covers the occurrence of the term “alkyl” where no numerical range is designated). The alkyl group of the compounds described herein may be designated as “C₁-C₆ alkyl” or similar designations. By way of example only, “C₁-C₆ alkyl” indicates that there are one to six carbon atoms in the alkyl chain, i.e., the alkyl chain is selected from the group consisting of methyl, ethyl, n-propyl, iso-propyl, n-butyl, iso-butyl, sec-butyl, t-butyl, n-pentyl, iso-pentyl, neo-pentyl, hexyl, propen-3-yl (allyl), cyclopropylmethyl, cyclobutylmethyl, cyclopentylmethyl, cyclohexylmethyl. Alkyl groups can be substituted or unsubstituted. Depending on the structure, an alkyl group can be a monoradical or a diradical (i.e., an alkylene group).

The term “alkenyl” refers to a type of alkyl group in which the first two atoms of the alkyl group form a double bond that is not part of an aromatic group. That is, an alkenyl group begins with the atoms —C(R)═CR₂, wherein R refers to the remaining portions of the alkenyl group, which may be the same or different. Non-limiting examples of an alkenyl group include —CH═CH₂, —C(CH₃)═CH₂, —CH═CHCH₃, —CH═C(CH₃)₂ and —C(CH₃)═CHCH₃. The alkenyl moiety may be branched, straight chain, or cyclic (in which case, it would also be known as a “cycloalkenyl” group). Alkenyl groups may have 2 to 6 carbons. Alkenyl groups can be substituted or unsubstituted. Depending on the structure, an alkenyl group can be a monoradical or a diradical (i.e., an alkenylene group).

The term “alkynyl” refers to a type of alkyl group in which the first two atoms of the alkyl group form a triple bond. That is, an alkynyl group begins with the atoms —C≡C—R, wherein R refers to the remaining portions of the alkynyl group. Non-limiting examples of an alkynyl group include —C≡CH, —C≡CCH₃, —C≡CCH₂CH₃ and —C≡CCH₂CH₂CH₃. The “R” portion of the alkynyl moiety may be branched, straight chain, or cyclic. An alkynyl group can have 2 to 6 carbons. Alkynyl groups can be substituted or unsubstituted. Depending on the structure, an alkynyl group can be a monoradical or a diradical (i.e., an alkynylene group).

“Carbocycle” refers to saturated, unsaturated or aromatic rings in which each atom of the ring is carbon. Carbocycle may be monocyclic or polycyclic and may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring. e.g., phenyl, may be fused to a saturated or unsaturated ring, e.g., cyclohexane, cyclopentane, or cy clohexene. Any combination of saturated, unsaturated and aromatic bicyclic rings, as valence permits, is included in the definition of carbocyclic.

Exemplary carbocycles include cyclopropyl, cyclobutyl, cyclopentyl, cyclohexyl, cyclohexenyl, adamantyl, phenyl, indanyl, and naphthyl. Unless stated otherwise specifically in the specification, a carbocycle is optionally substituted by one or more substituents such as those substituents described herein.

The term “trityl” refers to a triphenylmethyl group. In the art, “trityl” protecting groups are covalently attached to heteroatoms, and are used to protect heteroatoms from undesired chemical reactions.

The term “halo” or, alternatively, “halogen” means fluoro, chloro, bromo, or iodo.

The compounds disclosed herein, in some embodiments, are used in different enriched isotopic forms, e.g., enriched in the content of ²H, ³H, ¹¹C, ¹³C and/or ¹⁴C. In one particular embodiment, a compound described herein is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵, ³⁶S, ³⁵Cl, ³⁷Cl, ⁷⁹Br, ⁸¹Br, ¹²⁵I are all contemplated. All isotopic variations of the compounds described herein, whether radioactive or not, are encompassed within the scope of the present disclosure.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

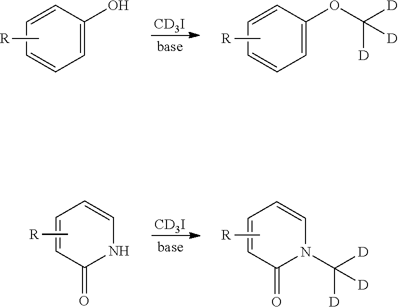

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

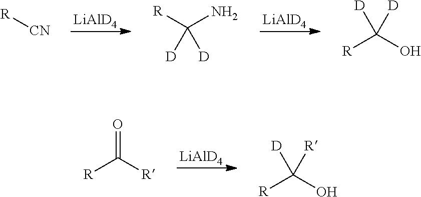

Deuterium gas and palladium catalysts are employed to reduce unsaturated carbon-carbon linkages and to perform a reductive substitution of aryl carbon-halogen bonds as illustrated, by way of example only, in the reaction schemes below.

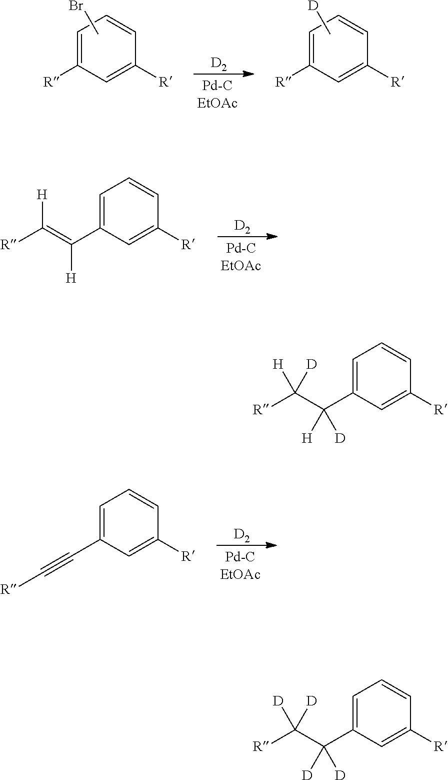

The term “tertiary amine base” refers to a nitrogen base that has exceeded its bonding valence. In the art, “tertiary amine bases” are also referred to as “bulky” or “non-nucleophilic” base, as they are less susceptible to nucleophilic attack. Examples of “tertiary amine base” as used herein include, but are not limited to, pyridine, triethylamine, triisopropyl amine, tributylamine, 2-tert-butyl-1,1,3,3-tetramethylguanidine, 4-dimethylaminopyridine, N,N-diisopropylethylamine, 1,8-diazabicycloundec-7-ene, 1,5-diazabicyclo(4.3.0)non-5-ene, 2,6-di-tert-butylpyridine, 1,8-bis(dimethylamino)naphthalene, 2,6-lutidine, 1,1,3,3-tetramethylguanidine, 2,2,6,6-tetramethylpiperidine, 2,4,6-trimethylpyridine, 1,4-diazabicyclo(2.2.2)octane, N,N-dicyclohexylmethylamine, quinuclidine, pempidine, 1,5,7-triazabicyclo(4.4.0)dec-5-ene, 7-methyl-1,5,7-triazabicyclo(4.4.0)dec-5-ene, 3,3,6,9,9-pentamethyl-2,10-diazabicyclo-(4.4.0)dec-1-ene, and N-methylmorpholine.

The term “aprotic polar solvent” refers to a solvent that lacks an acidic, or an exchangeable, hydrogen atom. Intrinsically, an “aprotic polar solvent” does not facilitate hydrogen bonding interactions, and facilitates S_{N}2-type reactions. Examples of “aprotic polar solvent” as used herein include, but are not limited to, chloroform. N-methylpyrrolidone, tetrahydrofuran, 2-methyltetrahydrofuran, ethyl acetate, acetone, N,N-dimethylformamide (dimethylformamide, or DMF), N,N-dimethylacetamide (dimethylacetamide, or DMA), acetonitrile (or MeCN), dimethyl sulfoxide (or DMSO), propylene carbonate, 1,4-dioxane (or dioxane), and dichloromethane (or DCM). The term “aprotic polar solvent” also encompasses mixtures, or combinations, of two or more aprotic polar solvents.

The term “protic polar solvent” refers to a solvent that has a labile, or an acidic, or an exchangeable, hydrogen atom. “Protic polar solvent” facilitate hydrogen bonding interactions. Examples of “protic polar solvent” as used herein include, but are not limited to, water, acetic acid, formic acid, methanol, ethanol, n-propanol, and t-butanol. The term “protic polar solvent” also encompasses mixtures, or combinations, of two or more protic polar solvents.

The term “polar solvent” refers to an aprotic polar solvent, or a protic polar solvent, or combinations thereof.

The term “acid” refers to a molecule that has a labile, or acidic, hydrogen atom. Examples of “acid” as used herein include, but are not limited to, trifluoroacetic acid (or TFA), 2, 2, 2-trifluoroethanol, sulfuric acid, nitric acid, hydrofluoric acid, hydrobromic acid, hydroiodic acid, triflic acid (or trifluoromethanesulfonic acid), perchloric acid, phosphoric acid, chloric acid, methanesulfonic acid, p-toluenesulfonic acid, acetic acid, formic acid, and hydrochloric acid.

Other examples of “acid” as used herein include, but are not limited to, molecules with a pKₐ measured in water less than about 5.5. The term “acid” also encompasses mixtures, or combinations, of two or more acids.

The term “base” refers to a molecule that can extract a hydrogen atom from another molecule. Examples of “base” as used herein include, but are not limited to, an alkali metal hydroxide, an alkali metal carbonate, an alkali metal bicarbonate, an alkali metal alkoxide, an alkali metal carboxylate, an alkali metal oxide, an alkali metal fluoride, an alkaline earth metal hydroxide, an alkaline earth metal carbonate, an alkaline earth metal bicarbonate, an alkaline earth metal alkoxide, an alkaline earth metal carboxylate, an alkaline earth metal oxide, a primary amine, a secondary amine, a tertiary amine, a lanthanide hydroxide, a lanthanide carbonate, a lanthanide bicarbonate, a lanthanide alkoxide, a lanthanide carboxylate, a lanthanide oxide, and combinations thereof. Representative, but not limiting, examples of “base” as used herein include lithium hydroxide, sodium hydroxide, potassium hydroxide, ammonium hydroxide, potassium acetate (KOAc), sodium acetate (NaOAc), tripotassium phosphate, sodium butoxide, potassium butoxide, potassium t-butoxide, sodium carbonate, potassium carbonate, cesium carbonate, cesium fluoride, sodium bicarbonate, potassium bicarbonate, calcium hydroxide, and triethylamine.

The term “hydrogenation” refers to a chemical reaction between molecular hydrogen and a reactant in the presence of a catalyst, such as, but not limited to a composition comprising nickel, palladium, platinum, rhodium, ruthenium, or combinations thereof. A “hydrogenation” reaction is commonly utilized to reduce or saturate organic compounds via the addition of hydrogen atom pairs.

The term “metal reduction” refers to a reduction where an alkali metal or low valent transition metal in a suitable solvent or solvent mixture adds the equivalent of hydrogen, two protons and two electrons, to a substrate molecule, resulting either in reductive cleavage of a single bond, or reduction of a multiple bond. In certain, but not all, cases a “metal reduction” as used herein is referred to as a “dissolving metal reduction” in the art.

The term “coupling reaction” refers to a chemical reaction where two fragments combine with the aid of a metal catalyst, or “coupling catalyst”. Examples of “coupling reactions” as used herein include, but are not limited to, reactions known in the art as “Suzuki”. “Negishi”, “Stille”, or “Liebeskind-Srogl” coupling reactions. Examples of “coupling catalysts” as used herein include, but are not limited, to a composition comprising copper, palladium, nickel, iron, or combinations thereof. The term “palladium-based catalyst” refers to a coupling catalyst comprising palladium. Examples of a “palladium-based catalyst” as used herein include, but are not limited to, Pd(PPh₃)₄, Pd(OAc)₂, Pd(dppf)Cl₂ (where“dppf” is 1,1′-bis(diphenylphosphino)ferrocene), Pd(dtbpf)Cl₂ (where “dtbpf” is 1,1′-bis(di-tert-butylphosphino)ferrocene, Pd(dba)₂ (bis(dibenzylideneacetone)palladium(0)), Pd₂(dba)₃ (tris-(dibenzylideneacetone)palladium(0)), Pd(PCy₃)₂ (where “Cy” is cyclohexyl), Pd(dppe)Cl₂ (where “dppe” is 1,2-bis(diphenylphosphino)ethane), Pd(t-Bu₃P)₂, PdCl₂[P(o-Tol)₃]₂, benzylbis-(triphenylphosphine)palladium(II) chloride, (A-Phos)₂Cl₂Pd, Na₂PdCl₄, Na₂PdCl₄/DTBPPS (where “DTBPPS” is 3-(di-tert-butylphosphonium)propane sulfonate), and PdCl₂(PPh₃)₄. Representative, but not limiting, examples of(A-Phos)₂Cl₂Pd palladium-based catalysts are disclosed in the Journal of Organic Chemistry 2007, 72, pages 5104-5112, by Guram et al.

The term “acyl halide preparation agent” refers to a chemical reagent that is used to convert a carboxylic acid or a carboxylic acid derivative, including, but not limited to, a carboxylic acid salt, to a carboxylic acid halide, or acyl halide. In the case where the halide is chloride, the “acyl halide preparation agent” is an “acyl chloride preparation agent”. Examples of “acyl chloride preparation agents”, as used herein include, but are not limited to oxalyl chloride, thionyl chloride, phosphoryl chloride, phosphorus trichloride, methanesulfonyl chloride, trichloromethanesulfonyl chloride, tert-butyl hypochlorite, dichloromethyl methyl ether, methoxyacetyl chloride, cyanuric chloride, N-chlorosuccinamide, N-chlorophthalimide, and trimethylsilyl chloride. In the case where the halide is bromide, the “acyl halide preparation agent” is an “acyl bromide preparation agent”. Examples of “acyl bromide preparation agents”, as used herein include, but are not limited to phosphorus tribromide, methanesulfonyl bromide, cyanuric bromide, triphenylphosphine/N-bromosuccinamide, and triphenylphosphine/bromine.

Compounds described herein may be formed as, and/or used as, pharmaceutically acceptable salts. The type of pharmaceutical acceptable salts, include, but are not limited to: (1) acid addition salts, formed by reacting the free base form of the compound with a pharmaceutically acceptable: inorganic acid, such as, for example, hydrochloric acid, hydrobromic acid, sulfuric acid, phosphoric acid, metaphosphoric acid, and the like; or with an organic acid, such as, for example, acetic acid, propionic acid, hexanoic acid, cyclopentanepropionic acid, glycolic acid, pyruvic acid, lactic acid, malonic acid, succinic acid, malic acid, maleic acid, fumaric acid, trifluoroacetic acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, toluenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, butyric acid, phenylacetic acid, phenylbutyric acid, valproic acid, and the like; (2) salts formed when an acidic proton present in the parent compound is replaced by a metal ion, e.g., an alkali metal ion (e.g. lithium, sodium, potassium), an alkaline earth ion (e.g. magnesium, or calcium), or an aluminum ion. In some cases, compounds described herein may coordinate with an organic base, such as, but not limited to, ethanolamine, diethanolamine, triethanolamine, tromethamine, N-methylglucamine, dicyclohexylamine, tris(hydroxymethyl)methylamine. In other cases, compounds described herein may form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, sodium hydroxide, and the like.

It should be understood that a reference to a pharmaceutically acceptable salt includes the solvent addition forms or crystal forms thereof, particularly solvates or polymorphs. Solvates contain either stoichiometric or non-stoichiometric amounts of a solvent, and may be formed during the process of crystallization with pharmaceutically acceptable solvents such as water, ethanol, and the like. Hydrates are formed when the solvent is water, or alcoholates are formed when the solvent is alcohol. Solvates of compounds described herein can be conveniently prepared or formed during the processes described herein. In addition, the compounds provided herein can exist in unsolvated as well as solvated forms. In general, the solvated forms are considered equivalent to the unsolvated forms for the purposes of the compounds and methods provided herein.

In addition, compounds described herein include crystalline forms, also known as polymorphs. Polymorphs include the different crystal packing arrangements of the same elemental composition of a compound. Polymorphs usually have different X-ray diffraction patterns, melting points, density, hardness, crystal shape, optical properties, stability, and solubility. Various factors such as the recrystallization solvent, rate of crystallization, and storage temperature may cause a single crystal form to dominate.

The synthetic method disclosed herein is an method for producing CRAC channel inhibitors. In some embodiments, this method produces kilogram quantities. The methods may improve previous synthetic routes by eliminating the presence of multiple undesirable impurities.

### Process of Synthesizing CRAC Channel Inhibitors

One aspect described herein is a process of synthesizing CRAC channel inhibitors. In some embodiments, the CRAC channel inhibitors are compounds of Formula (I):

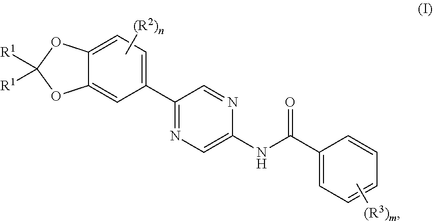

or pharmaceutically acceptable salts thereof, wherein:
R¹ is independently selected at each occurrence from hydrogen, halogen and C₁-C₃ alkyl optionally substituted with one or more substituents independently selected at each occurrence from halogen, —OR′, —CN, —N(R′)₂ and —NO₂;
R² and R³ are independently selected at each occurrence from halogen and C₁-C₃ alkyl optionally substituted with one or more substituents independently selected at each occurrence from halogen, —OR′, —CN, —N(R′)₂ and —NO₂:
or, when both R¹ are independently C₁-C₃ alkyl, the two R¹ groups are taken together with the atom to which they are attached to form a carbocycle;
n is 0, 1, 2 or 3;
m is 0, 1, 2, 3, 4, or 5: and
   R′ is independently selected at each occurrence from hydrogen; and C₁₋₆ alkyl, C₂₋₆ alkenyl, and C₂₋₆, alkynyl, each optionally substituted with one or more substituents independently selected at each occurrence from halogen, —CN, —NO₂, —OH, —NH₂, and OCH₃.

In some embodiments, R¹ is independently selected at each occurrence from hydrogen, halogen and C₁-C₃ alkyl optionally substituted with one or more substituents independently selected at each occurrence from halogen, —OH, —OCH₃, —CN, —NH₂, and —NO₂; and R² and R³ are independently selected at each occurrence from halogen and C₁-C₃ alkyl optionally substituted with one or more substituents independently selected at each occurrence from halogen, —OH, —OCH₃, —CN, —NH₂, and —NO₂.

One aspect described herein is a process of synthesizing CRAC channel inhibitors, wherein the CRAC channel inhibitors are compounds of Formulas (IA), (IB), (IC), (ID), (IE), (IF), or (IG):

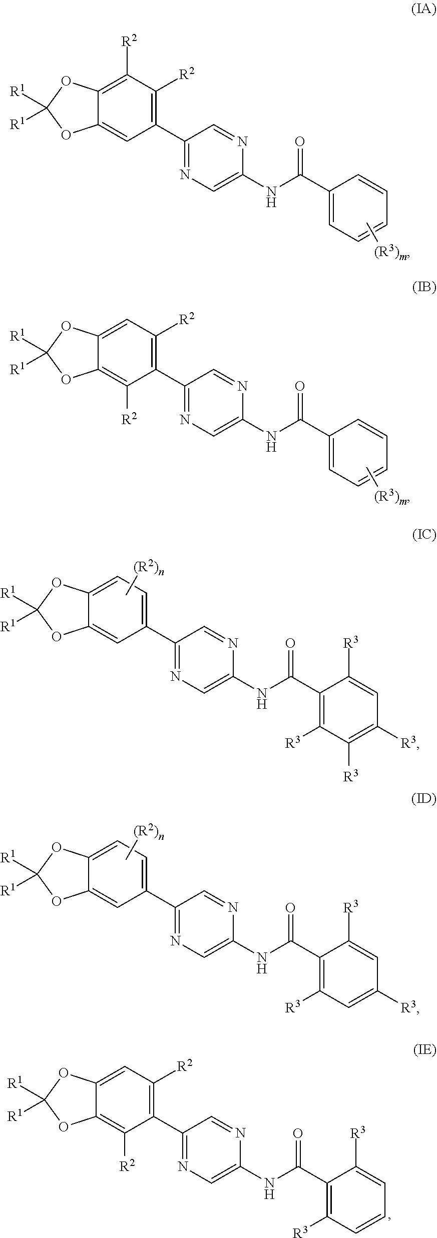

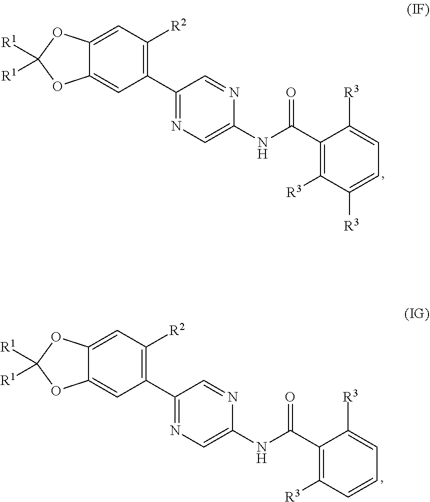

or a salt of any one thereof. In certain embodiments, for a compound, or salt, of any one of Formulas (I), (IA), (IB), (IC), (ID), (IE), (IF), and (IG), R¹ is independently selected at each occurrence from hydrogen, halogen and C₁-C₃ alkyl optionally substituted with one or more substituents independently selected at each occurrence from halogen, —OR′, —CN, —N(R′)₂ and —NO₂; R² and R³ are independently selected at each occurrence from halogen and C₁-C₃ alkyl optionally substituted with one or more substituents independently selected at each occurrence from halogen, —OR′, —CN, —N(R′)₂ and —NO₂; wherein R′ is independently selected at each occurrence from hydrogen: and C₁₋₆ alkyl, C₂₋₆ alkenyl, and C₂₋₆ alkynyl, each optionally substituted with one or more substituents independently selected at each occurrence from halogen, —CN, —NO₂, —OH, —NH₂, and OCH₃.

In some embodiments, R¹ is independently selected at each occurrence from hydrogen, halogen and C₁-C₃ alkyl optionally substituted with one or more substituents independently selected at each occurrence from halogen, —OH, —OCH₃, —CN, —NH₂, and —NO₂; and R² and R³ are independently selected at each occurrence from halogen and C₁-C₃ alkyl optionally substituted with one or more substituents independently selected at each occurrence from halogen, —OH, —OCH₃, —CN, —NH₂, and —NO₂.

In certain embodiments, for a compound or salt of any one of Formulas (I), (IA), (IB), (IC), and (ID), n is 0, 1, 2 or 3. In certain embodiments, for a compound or salt of any one of Formulas (I), (IA), (IB), (IC), and (ID), n is 0, 1, or 2. In certain embodiments, for a compound or salt of any one of Formulas (I), (IA), (IB), (IC), and (ID), n is 0 or 1. In certain embodiments, for a compound or salt of any one of Formulas (I), (IA), (IB), (IC), and (ID), n is 1. In embodiments, for a compound, or salt, of any one of Formulas (I), (IA), (IB), (IC), (ID), (IE), (IF), and (IG), where n is 0, 1, or 2, the open position(s), position(s) without R² on the aromatic ring, is(are) occupied by hydrogen.

In certain embodiments, for a compound or salt of any one of Formulas (I), (IA), and (IB), m is 0, 1, 2, 3, or 4. In certain embodiments, for a compound or salt of any one of Formulas (I), (IA), and (IB), m is 0, 1, 2, or 3. In certain embodiments, for a compound or salt of any one of Formulas (I), (IA), and (IB), m is 0, 1, or 2. In certain embodiments, for a compound or salt of any one of Formulas (I), (IA), and (IB), m is 2. In embodiments, for a compound, or salt, of any one of Formulas (I), (IA), (IB), (IC), (ID), (IE), (IF), and (IG), where m is 0, 1, 2, 3, or 4, the open position(s), position(s) without R³ on the aromatic ring, is(are) occupied by hydrogen.

In certain embodiments, for a compound, or salt, of any one of Formulas (I), (IE), (IF), and (IG), n is 1 or 2, m is 2 or 3, and the open positions, positions that are not substituted with R² or R³, are occupied by hydrogen, according to standard conventions applicable to structural drawings. In certain embodiments, for a compound, or salt, of any one of Formulas (I) and (IG), n is 1, m is 2, and the open positions, positions that are not substituted with R² or R³, are occupied by hydrogen, according to standard conventions applicable to structural drawings.

In certain embodiments, for a compound or salt of any one of Formulas (I), (IA), (IB), (IC), (ID), (IE), (IF), and (IG), R′ is independently selected at each occurrence from hydrogen; and C₁₋₆ alkyl, C₂₋₆ alkenyl, and C₂₋₄ alkynyl, each optionally substituted with one or more substituents independently selected at each occurrence from halogen, —CN, —NO₂, —OH, —NH₂, and OCH₃.

In some embodiments, for a compound, or salt, of Formula (IG), R¹ and R² are independently at each occurrence a halogen and R³ is independently at each occurrence a halogen or a C₁-C₃ alkyl. In some embodiments, for a compound, or salt, of Formula (IG), both R¹ are fluoro, R² is a halogen and R³ is independently at each occurrence a halogen or a C₁-C₃ alkyl. In some embodiments, for a compound, or salt, of Formula (IG), both R¹ are fluoro, R² is chloro or fluoro, and R³ is independently at each occurrence a halogen or a C₁-C₃ alkyl. In some embodiments, for a compound, or salt, of Formula(IG), both R¹ are fluoro, R² is chloro or fluoro, and R³ is independently at each occurrence a halogen, methyl, or ethyl. In some embodiments, for a compound, or salt, of Formula (IG), both R¹ are fluoro, R² is chloro or fluoro, and R³ is independently at each occurrence a halogen or methyl. In some embodiments, for a compound, or salt, of Formula (IG), both R¹ are fluoro, R² is chloro or fluoro, and R³ is independently at each occurrence chloro, fluoro, or methyl. In some embodiments, for a compound, or salt, of Formula (IG), both R¹ are fluoro, R² is chloro, and R³ is independently at each occurrence chloro, fluoro, or methyl. In some embodiments, for a compound, or salt, of Formula (IG), both R¹ are fluoro.

R² is chloro, and R³ is independently at each occurrence fluoro or methyl. In some embodiments, for a compound, or salt, of Formula (IG), both R¹ are fluoro, R² is chloro, one of R³ is fluoro, and one of R³ is methyl.

One aspect described herein is a process of synthesizing CRAC channel inhibitors. In some embodiments, the CRAC channel inhibitors are compounds of Formula (I), (IA), (IB), (IC), (ID), (IE), (IF), or (IG):

wherein the process comprises contacting a compound of Formula (I-A):

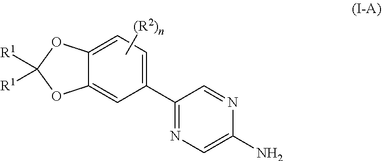

with a compound of Formula (I-B):

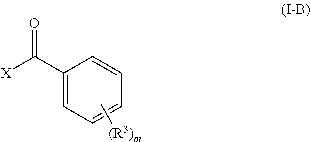

in the presence of a tertiary amine base and an aprotic polar solvent, wherein X is —Cl, —Br, —I, —CN, —N₃, —OCH₃, —OCH₂CH₃, —OC₆H₅, —OC₆H₄-4-NO₂, —OC(O)CH₃, —OC(O)C₆H₅, —O(SO₂)CH₃, or —O(SO₂)C₆H₄-4-CH₃.

In some embodiments, the tertiary amine base is selected from the group consisting of pyridine, triethylamine, triisopropyl amine, tributylamine, 2-tert-butyl-1,1,3,3-tetramethylguanidine, 4-dimethylaminopyridine, N,N-diisopropylethylamine, 1,8-diazabicycloundec-7-ene, 1,5-diazabicyclo(4.3.0)non-5-ene, 2,6-di-tert-butylpyridine, 1,8-bis(dimethylamino)naphthalene, 2,6-lutidine, 1,1,3,3-tetramethylguanidine, 2,2,6,6-tetramethylpiperidine, 2,4,6-trimethylpyridine, 1,4-diazabicyclo(2.2.2)octane, N,N-dicyclohexylmethylamine, quinuclidine, pempidine, 1,5,7-triazabicyclo(4.4.0)dec-5-ene, 7-methyl-1,5,7-triazabicyclo(4.4.0)dec-5-ene, 3,3,6,9,9-pentamethyl-2,10-diazabicyclo-(4.4.0)dec-1-ene, and N-methylmorpholine. In some embodiments, the tertiary amine base is selected from the group consisting of pyridine, triethylamine, triisopropyl amine, 2-tert-butyl-1,1,3,3-tetramethylguanidine, 4-dimethylaminopyridine, N,N-diisopropylethylamine and N-methylmorpholine. In some embodiments, the tertiary amine base is pyridine.

In some embodiments, the aprotic polar solvent is selected from the group consisting of chloroform, N-methylpyrrolidone, tetrahydrofuran, ethyl acetate, acetone, dimethylformamide, dimethylacetamide, acetonitrile, dimethyl sulfoxide, propylene carbonate, dichloromethane, and mixtures thereof. In some embodiments, the aprotic polar solvent is selected from the group consisting of chloroform, dichloromethane, and mixtures thereof. In some embodiments, the aprotic polar solvent is dichloromethane.

In some embodiments, the compound of Formula (I-A):

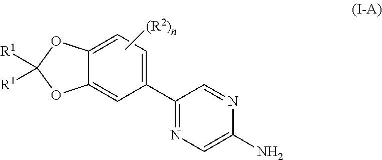

is synthesized by treating a compound of formula (I-C):

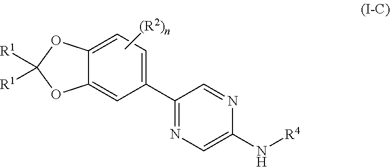

with an acid, wherein R⁴ is selected from the group consisting of trityl, t-butyl, t-butoxycarbonyl, p-tolyl, benzoyl, acetyl and benzyl.

In some embodiments, the acid is selected from the group consisting of trifluoroacetic acid, 2, 2, 2-trifluoroethanol, sulfuric acid, nitric acid, hydrofluoric acid, hydrobromic acid, hydroiodic acid, triflic acid, perchloric acid, phosphoric acid, chloric acid, methanesulfonic acid, p-toluenesulfonic acid, acetic acid, formic acid, and hydrochloric acid. In some embodiments, the acid is selected from the group consisting of trifluoroacetic acid, 2, 2, 2-trifluoroethanol, sulfuric acid, and hydrochloric acid. In some embodiments, the acid is hydrochloric acid.

In some embodiments, the compound of Formula (I-A):

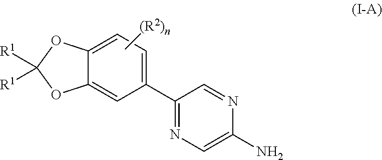

is synthesized by subjecting a compound of formula (I-C):

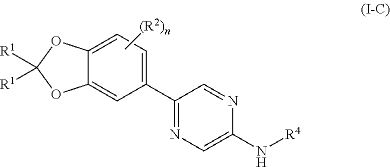

to a hydrogenation or metal reduction, wherein R⁴ is selected from the group consisting of trityl, t-butyl, p-tolyl, benzoyl, acetyl and benzyl. In some embodiments, R⁴ is selected from the group consisting of trityl, t-butyl, p-tolyl, and benzyl. In some embodiments, R⁴ is selected from the group consisting of trityl and benzyl. In some embodiments, R⁴ is benzyl. In some embodiments, R⁴ is trityl.

In some embodiments, the hydrogenation uses a metal catalyst selected from the group consisting of Ni, Raney Ni, Pd/C, Degussa type catalyst, Pt/C, and Pd(OAc)₂. In some embodiments, the hydrogenation uses a metal catalyst selected from the group consisting of Ni, Raney Ni, and Pd/C. In some embodiments, the hydrogenation uses a metal catalyst selected from the group consisting of Ni or Raney Ni. In some embodiments, the hydrogenation catalyst is Ni. In some embodiments, the hydrogenation catalyst is Raney Ni.

In some embodiments, the metal reduction uses a metal selected from the group consisting of lithium, sodium, and potassium, and the metal reduction optionally uses a catalyst. In some embodiments the catalyst is naphthalene. In some embodiments, the metal reduction uses a metal that is lithium and a catalyst that is naphthalene.

In some embodiments, the compound of formula (I-C):

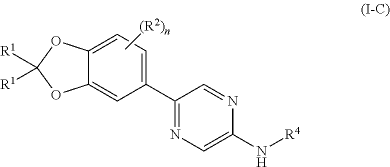

is synthesized by coupling a compound of formula (I-D):

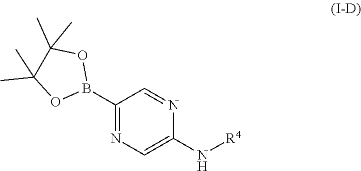

and a compound of formula (I-E):

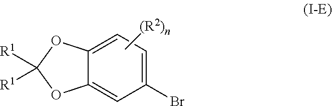

in the presence of a coupling catalyst.

In some embodiments, the compound of formula (I-C) is synthesized by coupling a compound of formula (I-D) and a compound of formula (I-E) in the presence of a coupling catalyst, a base, and a polar solvent.

In some embodiments, the compound of formula (I-C):

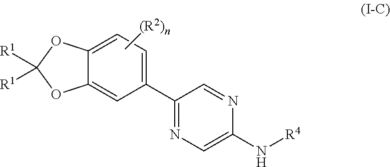

is synthesized by coupling a compound of formula (I-D):

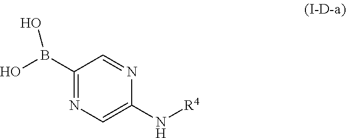

and a compound of formula (I-E):

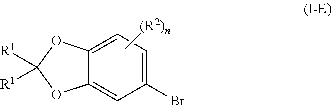

in the presence of a coupling catalyst.

In some embodiments, the compound of formula (I-C) is synthesized by coupling a compound of formula (I-D-a) and a compound of formula (I-E) in the presence of a coupling catalyst, a base, and a polar solvent.

In some embodiments, the coupling catalyst is a palladium-based catalyst. In some embodiments, the palladium-based catalyst is selected from the group consisting of Pd(PPh₃)₄, Pd(OAc)₂, Pd(dppf)Cl₂, Pd(dtbpf)Cl₂, Pd(dba)₂, Pd(PCy₃)₂, Pd(dppe)Cl₂, Pd(t-Bu₃P)₂, PdCl₂[P(o-Tol)₃]₂, benzylbis(triphenylphosphine)palladium(II) chloride, (A-Phos)₂Cl₂Pd, Na₂PdCl₄, SPhos (2-(2′,6″-dimethoxybiphenyl)dicyclohexylphosphine) and PdCl₂(PPh₃)₄. In some embodiments, the palladium-based catalyst is selected from the group consisting of Pd(PPh₃)₄, Pd(dppf)Cl₂ and PdCl₂(PPh₃)₄. In some embodiments, the palladium-based catalyst is Pd(PPh₃)₄.

In some embodiments, the base is selected from the group consisting of lithium hydroxide, sodium hydroxide, potassium hydroxide, ammonium hydroxide, potassium acetate, sodium acetate, tripotassium phosphate, sodium butoxide, potassium butoxide, sodium carbonate, potassium carbonate, cesium carbonate, sodium bicarbonate, potassium bicarbonate, calcium hydroxide, and triethylamine. In some embodiments, the base is selected from the group consisting of sodium acetate, potassium acetate, and tripotassium phosphate. In some embodiments, the base is potassium acetate.

In some embodiments, the polar solvent is selected from the group consisting of water, acetic acid, formic acid, methanol, ethanol, n-propanol, t-butanol, DMF, 1,4-dioxane, and combinations thereof. In some embodiments, the polar solvent comprises a combination of at least two of water, DMF, and 1,4-dioxane. In some embodiments, the polar solvent comprises a combination of water and DMF. In some embodiments, the polar solvent comprises a combination of DMF and 1,4-dioxane. In some embodiments, the polar solvent comprises a combination of water and 1,4-dioxane. In some embodiments, the polar solvent is 1,4-dioxane. In some embodiments, the polar solvent is DMF.

In some embodiments, the coupling reaction is conducted at a temperature from more than about 10° C., more than about 20° C., more than about 30° C., more than about 40° C., more than about 50° C., more than about 60° C., more than about 70° C., more than about 80° C., more than about 90° C., more than about 100° C. more than about 110° C., more than about 120° C. more than about 130° C., more than about 140° C., less than about 150T° C., less than about 140° C., less than about 130° C., less than about 120° C., less than about 110° C., less than about 100° C., less than about 90° C., less than about 80° C., less than about 70° C., less than about 60° C., less than about 50° C., less than about 40° C., less than about 30° C., less than about 20° C., from about 10° C. to about 150° C., from about 20° C. to about 140° C., from about 30° C. to about 130° C., from about 40° C. to about 120° C., from about 50° C. to about 110° C., from about 60° C. to about 110° C., from about 70° C. to about 100° C., from about 70° C. to about 90° C., from about 80° C. to about 90° C., from about 70° C. to about 80° C., from about 75° C. to about 85° C., or from about 85° C. to about 95° C.

In some embodiments, the compound of formula (I-D):

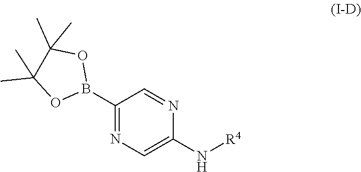

is synthesized by treating a compound of formula (I-F)

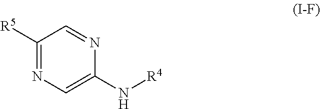

with a suitable boron-containing reagent in the presence of a palladium-based catalyst, a base, and a polar solvent, wherein R⁵ is independently selected from a halogen, —OTs (where “OTs” is O(SO₂)C₆H₄-4-CH₃), and —OMs (where “OMs” is O(SO₂)CH₃).

In some embodiments, the boron-containing reagent is a diboron agent. In some embodiments, the boron-containing reagent is bis(pinacolato)diboron. In some embodiments, the palladium-based catalyst is selected from the group consisting of Pd(PPh₃)₄, Pd(OAc)₂, Pd(dppf)Cl₂, Pd(dtbpf)Cl₂, Pd(dba)₂, Pd(PCy₃)₂, Pd(dppe)Cl₂, Pd(t-Bu₃P)₂, PdCl₂[P(o-Tol)₃]₂, benzylbis(triphenylphosphine)palladium(II) chloride, (A-Phos)₂Cl₂Pd, Na₂PdCl₄, and PdCl₂(PPh₃)₄. In some embodiments, the palladium-based catalyst is selected from the group consisting of Pd(PPh₃)₄, Pd(dppf)Cl₂ and PdCl₂(PPh₃)₄. In some embodiments, the palladium-based catalyst is Pd(dppf)Cl₂.

In some embodiments, the base is selected from the group consisting of lithium hydroxide, sodium hydroxide, potassium hydroxide, ammonium hydroxide, potassium acetate, sodium acetate, tripotassium phosphate, sodium butoxide, potassium butoxide, sodium carbonate, potassium carbonate, cesium carbonate, sodium bicarbonate, potassium bicarbonate, calcium hydroxide, and triethylamine. In some embodiments, the base is selected from the group consisting of sodium acetate, potassium acetate, and tripotassium phosphate. In some embodiments, the base is potassium acetate.

In some embodiments, the polar solvent is selected from the group consisting of water, acetic acid, formic acid, methanol, ethanol, n-propanol, r-butanol, and 1,4-dioxane. In some embodiments, the polar solvent is 1,4-dioxane.

In some embodiments, the compound of formula (I-F) is:

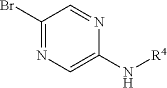

and is synthesized from 5-bromopyrazine-2-amine(or 2-amino-5-bromopyrazine).

In some embodiments, the compound of formula (I-F) is in a form selected from the group consisting of a solid, a liquid, and a solution. In some embodiments, the solid is a crystalline solid or an amorphous solid. In some embodiments, the solid is a crystalline solid.

In some embodiments, the compound of formula (I-B):

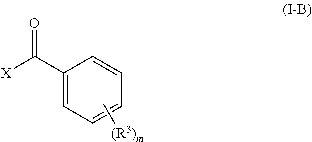

is synthesized by treating a compound of formula (I-G):

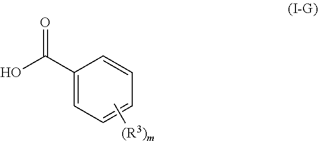

with an acyl halide preparation agent.

In some embodiments, the acyl halide preparation agent is selected from the group consisting of oxalyl chloride, thionyl chloride, phosphoryl chloride, phosphorus trichloride, methanesulfonyl chloride, trichloromethanesulfonyl chloride, tert-butyl hypochlorite, dichloromethyl methyl ether, methoxyacetyl chloride, cyanuric chloride, N-chlorosuccinamide, N-chlorophthalimide, and trimethylsilyl chloride. In some embodiments, the acyl halide preparation agent is selected from the group consisting of oxalyl chloride, thionyl chloride, phosphoryl chloride, and phosphorus trichloride. In some embodiments, the acyl halide preparation agent is oxalyl chloride.

One aspect described herein is a process of synthesizing CRAC channel inhibitors. In some embodiments, the CRAC channel inhibitors are compounds of Formula (IE):

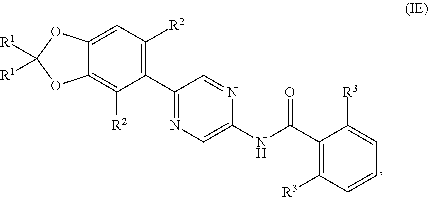

wherein the process comprises contacting a compound of Formula (IE-A):

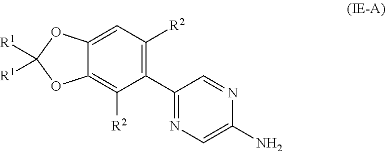

with a compound of Formula (IE-B):

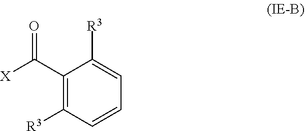

in the presence of a tertiary amine base and an aprotic polar solvent, wherein X is —Cl, —Br, —I, —CN, —N₃, —OCH₃, —OCH₂CH₃, —OC₆H₅, —OC₆H₄-4-NO₂, —OC(O)CH₃, —OC(O)C₆H₅, —O(SO₂)CH₃, or —O(SO₂)C₆H₄-4-CH₃.

In some embodiments, the tertiary amine base is selected from the group consisting of pyridine, triethylamine, triisopropyl amine, tributylamine, 2-tert-butyl-1,1,3,3-tetramethylguanidine, 4-dimethylaminopyridine, N,N-diisopropylethylamine, 1,8-diazabicycloundec-7-ene, 1,5-diazabicyclo(4.3.0)non-5-ene,2,6-di-tert-butylpyridine, 1,8-bis(dimethylamino)naphthalene, 2,6-lutidine, 1,1,3,3-tetramethylguanidine, 2,2,6,6-tetramethylpiperidine, 2,4,6-trimethylpyridine, 1,4-diazabicyclo(2.2.2)octane, N,N-dicyclohexylmethylamine, quinuclidine, pempidine, 1,5,7-triazabicyclo(4.4.0)dec-5-ene, 7-methyl-1,5,7-triazabicyclo(4.4.0)dec-5-ene, 3,3,6,9,9-pentamethyl-2,10-diazabicyclo-(4.4.0)dec-1-ene, and N-methylmorpholine. In some embodiments, the tertiary amine base is selected from the group consisting of pyridine, triethylamine, triisopropyl amine, 2-tert-butyl-1,1,3,3-tetramethylguanidine, 4-dimethylaminopyridine, N,N-diisopropylethylamine and N-methylmorpholine. In some embodiments, the tertiary amine base is pyridine.

In some embodiments, the aprotic polar solvent is selected from the group consisting of chloroform, N-methylpyrrolidone, tetrahydrofuran, ethyl acetate, acetone, dimethylformamide, dimethylacetamide, acetonitrile, dimethyl sulfoxide, propylene carbonate, dichloromethane, and mixtures thereof. In some embodiments, the aprotic polar solvent is selected from the group consisting of chloroform, dichloromethane, and mixtures thereof. In some embodiments, the aprotic polar solvent is dichloromethane.

In some embodiments, the compound of Formula (IE-A):

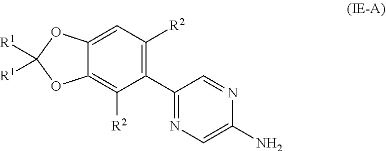

is synthesized by treating a compound of formula (IE-C):

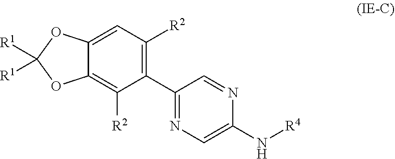

with an acid, wherein R⁴ is selected from the group consisting of trityl, t-butyl, t-butoxycarbonyl, p-tolyl, benzoyl, acetyl and benzyl.

In some embodiments, the acid is selected from the group consisting of trifluoroacetic acid, 2, 2, 2-trifluoroethanol, sulfuric acid, nitric acid, hydrofluoric acid, hydrobromic acid, hydroiodic acid, triflic acid, perchloric acid, phosphoric acid, chloric acid, methanesulfonic acid, p-toluenesulfonic acid, acetic acid, formic acid, and hydrochloric acid. In some embodiments, the acid is selected from the group consisting of trifluoroacetic acid, 2, 2, 2-trifluoroethanol, sulfuric acid, and hydrochloric acid. In some embodiments, the acid is hydrochloric.

In some embodiments, the compound of Formula (IE-A):

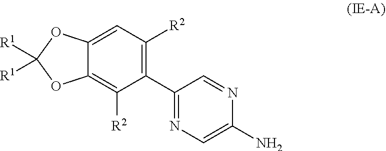

is synthesized by subjecting a compound of formula (IE-C):

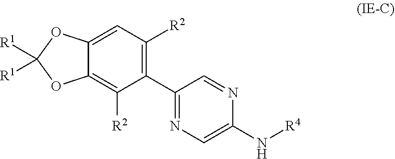

to a hydrogenation or metal reduction, wherein R⁴ is selected from the group consisting of trityl, t-butyl, p-tolyl, benzoyl, acetyl and benzyl. In some embodiments, R⁴ is selected from the group consisting of trityl, t-butyl, p-tolyl, and benzyl. In some embodiments, R⁴ is selected from the group consisting of trityl and benzyl. In some embodiments. R⁴ is benzyl. In some embodiments, R⁴ is trityl.

In some embodiments, the hydrogenation uses a metal catalyst selected from the group consisting of Ni, Raney Ni, Pd/C, Degussa type catalyst, Pt/C, and Pd(OAc)₂. In some embodiments, the hydrogenation uses a metal catalyst selected from the group consisting of Ni, Raney Ni, and Pd/C. In some embodiments, the hydrogenation uses a metal catalyst selected from the group consisting of Ni or Raney Ni. In some embodiments, the hydrogenation catalyst is Ni. In some embodiments, the hydrogenation catalyst is Raney Ni.

In some embodiments, the metal reduction uses a metal selected from the group consisting of lithium, sodium, and potassium, and the metal reduction optionally uses a catalyst.

In some embodiments the catalyst is naphthalene. In some embodiments, the metal reduction uses a metal that is lithium and a catalyst that is naphthalene.

In some embodiments, the compound of formula (IE-C):

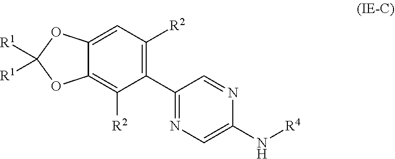

is synthesized by coupling a compound of formula (IE-D):

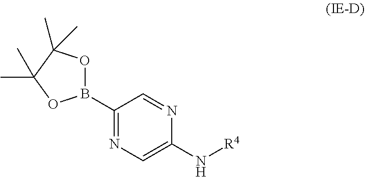

and a compound of formula (IE-E):

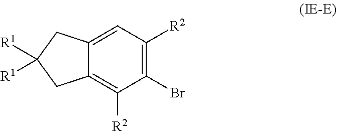

in the presence of a coupling catalyst.

In some embodiments, the compound of formula (IE-C) is synthesized by coupling a compound of formula (IE-D) and a compound of formula (IE-E) in the presence of a coupling catalyst, a base, and a polar solvent.

In some embodiments, the coupling catalyst is a palladium-based catalyst. In some embodiments, the palladium-based catalyst is selected from the group consisting of Pd(PPh₃)₄, Pd(OAc)₂, Pd(dppf)Cl₂, Pd(dtbpf)Cl₂, Pd(dba)₂, Pd(PCy₃)₂, Pd(dppe)Cl₂, Pd(t-Bu₃P)₂, PdCl₂[P(o-Tol)₃]₂, benzylbis(triphenylphosphine)palladium(II) chloride, (A-Phos)₂Cl₂Pd, Na₂PdCl₄, SPhos (2-(2′,6″-dimethoxybiphenyl)dicyclohexylphospine), and PdCl₂(PPh₃)₄. In some embodiments, the palladium-based catalyst is selected from the group consisting of Pd(PPh₃)₄, Pd(dppf)Cl₂ and PdCl₂(PPh₃)₄. In some embodiments, the palladium-based catalyst is Pd(PPh₃)₄.

In some embodiments, the base is selected from the group consisting of lithium hydroxide, sodium hydroxide, potassium hydroxide, ammonium hydroxide, potassium acetate, sodium acetate, tripotassium phosphate, sodium butoxide, potassium butoxide, sodium carbonate, potassium carbonate, cesium carbonate, sodium bicarbonate, potassium bicarbonate, calcium hydroxide, and triethylamine. In some embodiments, the base is selected from the group consisting of sodium acetate, potassium acetate, and tripotassium phosphate. In some embodiments, the base is potassium acetate.

In some embodiments, the polar solvent is selected from the group consisting of water, acetic acid, formic acid, methanol, ethanol, n-propanol, t-butanol, DMF, 1,4-dioxane, and combinations thereof. In some embodiments, the polar solvent comprises a combination of at least two of water, DMF, and 1,4-dioxane. In some embodiments, the polar solvent comprises a combination of water and DMF. In some embodiments, the polar solvent comprises a combination of DMF and 1,4-dioxane. In some embodiments, the polar solvent comprises a combination of water and 1,4-dioxane. In some embodiments, the polar solvent is 1,4-dioxane. In some embodiments, the polar solvent is DMF.

In some embodiments, the coupling reaction is conducted at a temperature from more than about 10° C., more than about 20° C., more than about 30° C., more than about 40° C., more than about 50° C., more than about 60° C., more than about 70° C., more than about 80° C., more than about 90° C., more than about 100° C., more than about 110° C., more than about 120° C., more than about 130° C., more than about 140° C., less than about 150° C., less than about 140° C., less than about 130° C., less than about 120° C., less than about 110° C., less than about 100° C., less than about 90° C. less than about 80° C. less than about 70° C., less than about 60° C., less than about 50° C., less than about 40° C., less than about 30° C., less than about 20° C. from about 10° C. to about 150° C. from about 20° C. to about 140° C., from about 30° C. to about 130° C., from about 40° C. to about 120° C. from about 50° C. to about 110° C. from about 60° C. to about 110° C., from about 70° C. to about 100° C., from about 70° C. to about 90° C. from about 80° C. to about 90° C., from about 70° C. to about 80° C., from about 75° C. to about 85° C., or from about 85° C. to about 95° C.

In some embodiments, the compound of formula (IE-D):

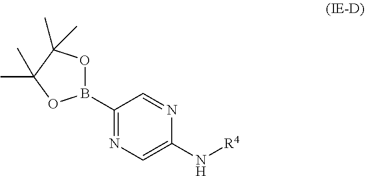

is synthesized by treating a compound of formula (IE-F)

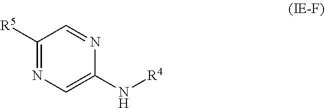

with a suitable boron-containing reagent in the presence of a palladium-based catalyst, a base, and a polar solvent, wherein R⁵ is independently selected from a halogen, OTs, and OMs.

In some embodiments, the boron-containing reagent is a diboron agent. In some embodiments, the boron-containing reagent is bis(pinacolato)diboron. In some embodiments, the palladium-based catalyst is selected from the group consisting of Pd(PPh₃)₄, Pd(OAc)₂, Pd(dppf)Cl₂, Pd(dtbpf)Cl₂, Pd(dba)₂, Pd(PCy₃)₂, Pd(dppe)Cl₂, Pd(t-Bu₃P)₂, PdCl₂[P(o-Tol)₃]₂, benzylbis(triphenylphosphine)palladium(II) chloride, (A-Phos)₂Cl₂Pd, Na₂PdCl₄, and PdCl₂(PPh₃)₄. In some embodiments, the palladium-based catalyst is selected from the group consisting of Pd(PPh₃)₄, Pd(dppf)Cl₂ and PdCl₂(PPh₃)₄. In some embodiments, the palladium-based catalyst is Pd(dppf)Cl₂.

In some embodiments, the base is selected from the group consisting of lithium hydroxide, sodium hydroxide, potassium hydroxide, ammonium hydroxide, potassium acetate, sodium acetate, tripotassium phosphate, sodium butoxide, potassium butoxide, sodium carbonate, potassium carbonate, cesium carbonate, sodium bicarbonate, potassium bicarbonate, calcium hydroxide, and triethylamine. In some embodiments, the base is selected from the group consisting of sodium acetate, potassium acetate, and tripotassium phosphate. In some embodiments, the base is potassium acetate.

In some embodiments, the polar solvent is selected from the group consisting of water, acetic acid, formic acid, methanol, ethanol, n-propanol, t-butanol, and 1,4-dioxane. In some embodiments, the polar solvent is 1,4-dioxane.

In some embodiments, the compound of formula (IE-F):

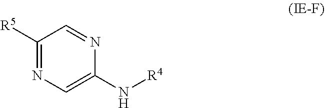

is formed by protecting 5-bromopyrazine-2-amine.

In some embodiments, the compound of formula (IE-F) is in a form selected from the group consisting of a solid, a liquid, and a solution. In some embodiments, the solid is a crystalline solid or an amorphous solid. In some embodiments, the solid is a crystalline solid.

In some embodiments, the compound of formula (IE-B):

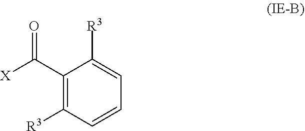

is synthesized by treating a compound of formula (IE-G):

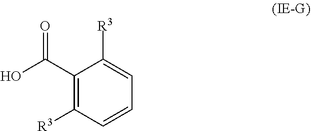

with an acyl halide preparation agent.

In some embodiments, the acyl halide preparation agent is selected from the group consisting of oxalyl chloride, thionyl chloride, phosphoryl chloride, phosphorus trichloride, methanesulfonyl chloride, trichloromethanesulfonyl chloride, tert-butyl hypochlorite, dichloromethyl methyl ether, methoxyacetyl chloride, cyanuric chloride, N-chlorosuccinamide, N-chlorophthalimide, and trimethylsilyl chloride. In some embodiments, the acyl halide preparation agent is selected from the group consisting of oxalyl chloride, thionyl chloride, phosphoryl chloride, and phosphorus trichloride. In some embodiments, the acyl halide preparation agent is oxalyl chloride.

One aspect described herein is a process of synthesizing CRAC channel inhibitors. In some embodiments, the CRAC channel inhibitors are compounds of Formula (IG);

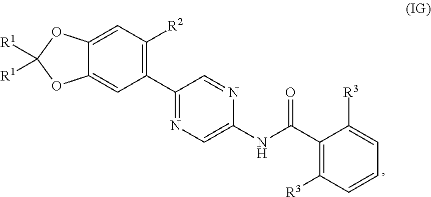

wherein the process comprises contacting a compound of Formula (IG-A):

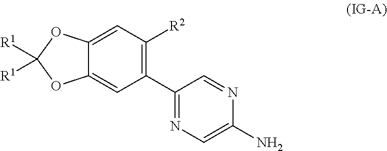

with a compound of Formula (IG-B):

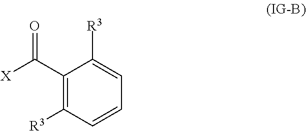

in the presence of a tertiary amine base and an aprotic polar solvent, wherein X is —Cl, —Br, —I, —CN, —N₃, —OCH₃, —OCH₂CH₃, —OC₆H₅, —OC₆H₄-4-NO₂, —OC(O)CH₃, —OC(O)C₆H₅, —O(SO₂)CH₃, or —O(SO₂)C₆H₄-4-CH₃.

In some embodiments, the tertiary amine base is selected from the group consisting of pyridine, triethylamine, triisopropyl amine, tributylamine, 2-tert-butyl-1,1,3,3-tetramethylguanidine, 4-dimethylaminopyridine, N,N-diisopropylethylamine, 1,8-diazabicycloundec-7-ene, 1,5-diazabicyclo(4.3.0)non-5-ene,2,6-di-tert-butylpyridine, 1,8-bis(dimethylamino)naphthalene, 2,6-lutidine, 1,1,3,3-tetramethylguanidine, 2,2,6,6-tetramethylpiperidine, 2,4,6-trimethylpyridine, 1,4-diazabicyclo(2.2.2)octane, N,N-dicyclohexylmethylamine, quinuclidine, pempidine, 1,5,7-triazabicyclo(4.4.0)dec-5-ene, 7-methyl-1,5,7-triazabicyclo(4.4.0)dec-5-ene, 3,3,6,9,9-pentamethyl-2,10-diazabicyclo-(4.4.0)dec-1-ene, and N-methylmorpholine. In some embodiments, the tertiary amine base is selected from the group consisting of pyridine, triethylamine, triisopropyl amine, 2-tert-butyl-1,1,3,3-tetramethylguanidine, 4-dimethylaminopyridine, N,N-diisopropylethylamine and N-methylmorpholine. In some embodiments, the tertiary amine base is pyridine.

In some embodiments, the aprotic polar solvent is selected from the group consisting of chloroform, N-methylpyrrolidone, tetrahydrofuran, ethyl acetate, acetone, dimethylformamide, dimethylacetamide, acetonitrile, dimethyl sulfoxide, propylene carbonate, dichloromethane, and mixtures thereof. In some embodiments, the aprotic polar solvent is selected from the group consisting of chloroform, dichloromethane, and mixtures thereof. In some embodiments, the aprotic polar solvent is dichloromethane.

In some embodiments, the compound of Formula (IG-A):

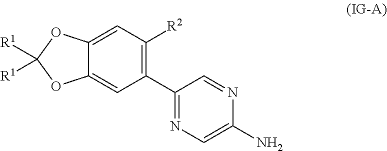

is synthesized by treating a compound of formula (IG-C):

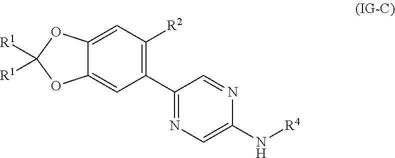

with an acid, wherein R⁴ is selected from the group consisting of trityl, t-butyl, t-butoxycarbonyl, p-tolyl, benzoyl, acetyl and benzyl.

In some embodiments, the acid is selected from the group consisting of trifluoroacetic acid, 2, 2, 2-trifluoroethanol, sulfuric acid, nitric acid, hydrofluoric acid, hydrobromic acid, hydroiodic acid, triflic acid, perchloric acid, phosphoric acid, chloric acid, methanesulfonic acid, p-toluenesulfonic acid, acetic acid, formic acid, and hydrochloric acid. In some embodiments, the acid is selected from the group consisting of trifluoroacetic acid, 2, 2, 2-trifluoroethanol, sulfuric acid, and hydrochloric acid. In some embodiments, the acid is hydrochloric.

In some embodiments, the compound of Formula (IG-A):

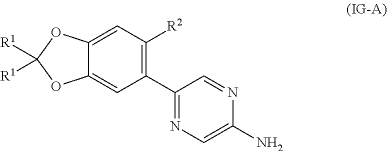

is synthesized by subjecting a compound of formula (IG-C):

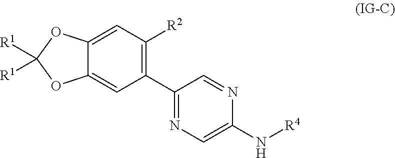

to a hydrogenation or metal reduction, wherein R⁴ is selected from the group consisting of trityl, t-butyl, p-tolyl, benzoyl, acetyl and benzyl. In some embodiments, R⁴ is selected from the group consisting of trityl, t-butyl, p-tolyl, and benzyl. In some embodiments, R⁴ is selected from the group consisting of trityl and benzyl. In some embodiments, R⁴ is benzyl. In some embodiments, R⁴ is trityl.

In some embodiments, the hydrogenation uses a metal catalyst selected from the group consisting of Ni, Raney Ni, Pd/C, Degussa type catalyst. Pt/C, and Pd(OAc)₂. In some embodiments, the hydrogenation uses a metal catalyst selected from the group consisting of Ni, Raney Ni, and Pd/C. In some embodiments, the hydrogenation uses a metal catalyst selected from the group consisting of Ni or Raney Ni. In some embodiments, the hydrogenation catalyst is Ni. In some embodiments, the hydrogenation catalyst is Raney Ni.

In some embodiments, the metal reduction uses a metal selected from the group consisting of lithium, sodium, and potassium, and the metal reduction optionally uses a catalyst. In some embodiments the catalyst is naphthalene. In some embodiments, the metal reduction uses a metal that is lithium and a catalyst that is naphthalene.

In some embodiments, the compound of formula (IG-C):

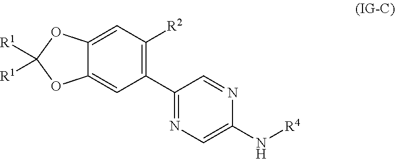

is synthesized by coupling a compound of formula (IG-D

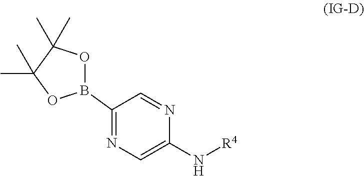

and a compound of formula (IG-E):

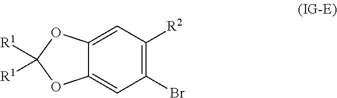

in the presence of a coupling catalyst.

In some embodiments, the compound of formula (IG-C) is synthesized by coupling a compound of formula (IG-D) and a compound of formula (IG-E) in the presence of a coupling catalyst, a base, and a polar solvent.

In some embodiments, the coupling catalyst is a palladium-based catalyst. In some embodiments, the palladium-based catalyst is selected from the group consisting of Pd(PPh₃)₄, Pd(OAc)₂, Pd(dppf)Cl₂, Pd(dtbpf)Cl₂, Pd(dba)₂, Pd(PCy₃)₂, Pd(dppe)Cl₂, Pd(t-Bu₃P)₂, PdCl₂[P(o-Tol)₃]₂, benzylbis(triphenylphosphine)palladium(II) chloride, (A-Phos)₂Cl₂Pd, Na₂PdCl₄, SPhos (2-(2′,6″-dimethoxybiphenyl)dicyclohexylphospine), and PdCl₂(PPh₃)₄. In some embodiments, the palladium-based catalyst is selected from the group consisting of Pd(PPh₃)₄, Pd(dppf)Cl₂ and PdCl₂(PPh₃)₄. In some embodiments, the palladium-based catalyst is Pd(PPh₃)₄.

In some embodiments, the base is selected from the group consisting of lithium hydroxide, sodium hydroxide, potassium hydroxide, ammonium hydroxide, potassium acetate, sodium acetate, tripotassium phosphate, sodium butoxide, potassium butoxide, sodium carbonate, potassium carbonate, cesium carbonate, sodium bicarbonate, potassium bicarbonate, calcium hydroxide, and triethylamine. In some embodiments, the base is selected from the group consisting of sodium acetate, potassium acetate, and tripotassium phosphate. In some embodiments, the base is potassium acetate.

In some embodiments, the polar solvent is selected from the group consisting of water, acetic acid, formic acid, methanol, ethanol, n-propanol, t-butanol, DMF, 1,4-dioxane, and combinations thereof. In some embodiments, the polar solvent comprises a combination of at least two of water, DMF, and 1,4-dioxane. In some embodiments, the polar solvent comprises a combination of water and DMF. In some embodiments, the polar solvent comprises a combination of DMF and 1,4-dioxane. In some embodiments, the polar solvent comprises a combination of water and 1,4-dioxane. In some embodiments, the polar solvent is 1,4-dioxane. In some embodiments, the polar solvent is DMF.

In some embodiments, the coupling reaction is conducted at a temperature from more than about 10° C., more than about 20° C. more than about 30° C., more than about 40° C., more than about 50° C., more than about 60°, more than about 70° C., more than about 80° C., more than about 90° C., more than about 1000 (, more than about 110° C., more than about 120° C., more than about 130° C., more than about 140° C. less than about 150° C., less than about 1400(2, less than about 130° C., less than about 120° C., less than about 110° C., less than about 100°, less than about 90° C. less than about 80° C. less than about 70° C., less than about 60° C., less than about 50° C., less than about 40° C., less than about 30° C., less than about 20° C., from about 10° C. to about 150° C. from about 200° C. to about 140° C., from about 30° C. to about 130° C., from about 40° C. to about 120° C., from about 50T° C. to about 110° C., from about 60° C. to about 110° C., from about 70° C. to about 100° C., from about 70° C. to about 90° C. from about 80° C. to about 90° C., from about 70° C. to about 80° C., from about 75° C. to about 85° C., or from about 85° C. to about 95° C.

In some embodiments, the compound of formula (IG-D):

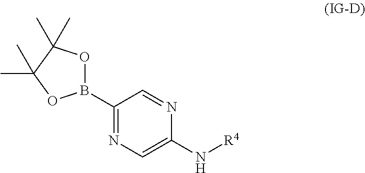

is synthesized by treating a compound of formula (IG-F)

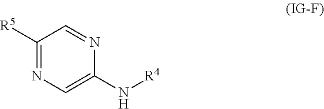

with a suitable boron-containing reagent in the presence of a palladium-based catalyst, a base, and a polar solvent, wherein R⁵ is independently selected from a halogen, OTs, and OMs.

In some embodiments, the boron-containing reagent is a diboron agent. In some embodiments, the boron-containing reagent is bis(pinacolato)diboron. In some embodiments, the palladium-based catalyst is selected from the group consisting of Pd(PPh₃)₄, Pd(OAc)₂, Pd(dppf)Cl₂, Pd(dtbpf)Cl₂, Pd(dba)₂, Pd(PCy₃)₂, Pd(dppe)C₂, Pd(t-Bu₃P)₂, PdCl₂[P(o-Tol)₃]₂, benzylbis(triphenylphosphine)palladium(II) chloride, (A-Phos)₂Cl₂Pd, Na₂PdCl₄, and PdCl₂(PPh₃)₄. In some embodiments, the palladium-based catalyst is selected from the group consisting of Pd(PPh₃)₄, Pd(dppf)Cl₂ and PdCl₂(PPh₃)₄. In some embodiments, the palladium-based catalyst is Pd(dppf)Cl₂.

In some embodiments, the base is selected from the group consisting of lithium hydroxide, sodium hydroxide, potassium hydroxide, ammonium hydroxide, potassium acetate, sodium acetate, tripotassium phosphate, sodium butoxide, potassium butoxide, sodium carbonate, potassium carbonate, cesium carbonate, sodium bicarbonate, potassium bicarbonate, calcium hydroxide, and triethylamine. In some embodiments, the base is selected from the group consisting of sodium acetate, potassium acetate, and tripotassium phosphate. In some embodiments, the base is potassium acetate.

In some embodiments, the polar solvent is selected from the group consisting of water, acetic acid, formic acid, methanol, ethanol, n-propanol, t-butanol, and 1,4-dioxane. In some embodiments, the polar solvent is 1,4-dioxane.

In some embodiments, the compound of formula (IG-F):

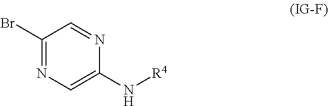

is formed by protecting 5-bromopyrazine-2-amine.

In some embodiments, the compound of formula (IG-F) is in a form selected from the group consisting of a solid, a liquid, and a solution. In some embodiments, the solid is a crystalline solid or an amorphous solid. In some embodiments, the solid is a crystalline solid.

In some embodiments, the compound of formula (IG-B).

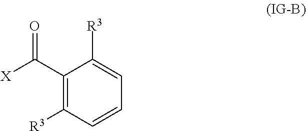

is synthesized by treating a compound of formula (IG-G):

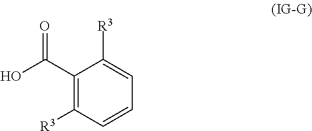

with an acyl halide preparation agent.

In some embodiments, the acyl halide preparation agent is selected from the group consisting of oxalyl chloride, thionyl chloride, phosphoryl chloride, phosphorus trichloride, methanesulfonyl chloride, trichloromethanesulfonyl chloride, tert-butyl hypochlorite, dichloromethyl methyl ether, methoxyacetyl chloride, cyanuric chloride, N-chlorosuccinamide, N-chlorophthalimide, and trimethylsilyl chloride. In some embodiments, the acyl halide preparation agent is selected from the group consisting of oxalyl chloride, thionyl chloride, phosphoryl chloride, and phosphorus trichloride. In some embodiments, the acyl halide preparation agent is oxalyl chloride.

One aspect described herein is a process of synthesizing a CRAC channel inhibitor, wherein the CRAC channel inhibitor is a compound of Formula (II):

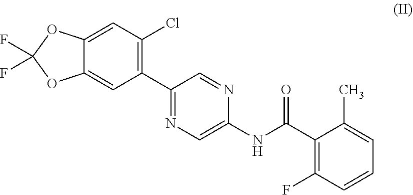 [SMILES: Cc1cccc(F)c1C(=O)Nc1cnc(-c2cc3c(cc2Cl)OC(F)(F)O3)cn1]

and, wherein the process comprises contacting a compound of Formula (II-A):

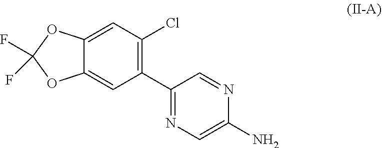 [SMILES: Nc1cnc(-c2cc3c(cc2Cl)OC(F)(F)O3)cn1]

with a compound of Formula (II-B):

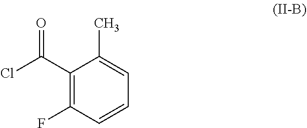

in the presence of a tertiary amine base and an aprotic polar solvent.

In some embodiments, the tertiary amine base is selected from the group consisting of pyridine, triethylamine, triisopropyl amine, tributylamine, 2-tert-butyl-1,1,3,3-tetramethylguanidine, 4-dimethylaminopyridine, N,N-diisopropylethylamine, 1,8-diazabicycloundec-7-ene, 1,5-diazabicyclo(4.3.0)non-5-ene, 2,6-di-tert-butylpyridine, 1,8-bis(dimethylamino)naphthalene, 2,6-lutidine, 1,1,3,3-tetramethylguanidine, 2,2,6,6-tetramethylpiperidine, 2,4,6-trimethylpyridine, 1,4-diazabicyclo(2.2.2)octane, N,N-dicyclohexylmethylamine, quinuclidine, pempidine, 1,5,7-triazabicyclo(4.4.0)dec-5-ene, 7-methyl-1,5,7-triazabicyclo(4.4.0)dec-5-ene, 3,3,6,9,9-pentamethyl-2,10-diazabicyclo-(4.4.0)dec-1-ene, and N-methylmorpholine. In some embodiments, the tertiary amine base is selected from the group consisting of pyridine, triethylamine, triisopropyl amine, 2-tert-butyl-1,1,3,3-tetramethylguanidine, 4-dimethylaminopyridine, N,N-diisopropylethylamine and N-methylmorpholine. In some embodiments, the tertiary amine base is pyridine.

In some embodiments, the aprotic polar solvent is selected from the group consisting of chloroform, N-methylpyrrolidone, tetrahydrofuran, ethyl acetate, acetone, dimethylformamide, dimethylacetamide, acetonitrile, dimethyl sulfoxide, propylene carbonate, dichloromethane, and mixtures thereof. In some embodiments, the aprotic polar solvent is selected from the group consisting of chloroform, dichloromethane, and mixtures thereof. In some embodiments, the aprotic polar solvent is dichloromethane.

In some embodiments, wherein the compound of Formula (II-A):

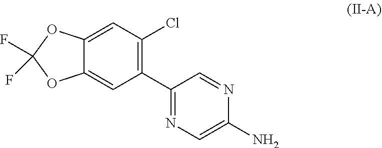 [SMILES: Nc1cnc(-c2cc3c(cc2Cl)OC(F)(F)O3)cn1]

is synthesized by treating a compound of formula (II-C):

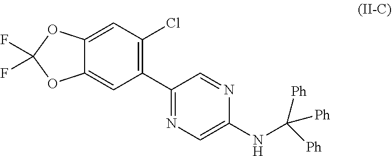 [SMILES: FC1(F)Oc2cc(Cl)c(-c3cnc(NC(c4ccccc4)(c4ccccc4)c4ccccc4)cn3)cc2O1]

with an acid.

In some embodiments, the acid is selected from the group consisting of trifluoroacetic acid, 2, 2, 2-trifluoroethanol, sulfuric acid, nitric acid, hydrofluoric acid, hydrobromic acid, hydroiodic acid, triflic acid, perchloric acid, phosphoric acid, chloric acid, methanesulfonic acid, p-toluenesulfonic acid, acetic acid, formic acid, and hydrochloric acid. In some embodiments, the acid is selected from the group consisting of trifluoroacetic acid, 2, 2, 2-trifluoroethanol, sulfuric acid, and hydrochloric acid. In some embodiments, the acid is hydrochloric acid.

In some embodiments, the compound of Formula (II-A):

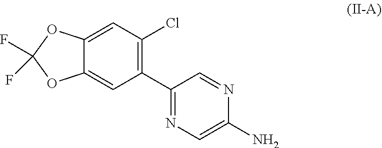 [SMILES: Nc1cnc(-c2cc3c(cc2Cl)OC(F)(F)O3)cn1]

is synthesized by subjecting a compound of formula (II-C):

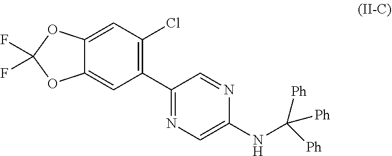 [SMILES: FC1(F)Oc2cc(Cl)c(-c3cnc(NC(c4ccccc4)(c4ccccc4)c4ccccc4)cn3)cc2O1]

to a hydrogenation or metal reduction.

In some embodiments, the hydrogenation uses a metal catalyst selected from the group consisting of Ni, Raney Ni, Pd/C, Degussa type catalyst, Pt/C, and Pd(OAc)₂. In some embodiments, the hydrogenation uses a metal catalyst selected from the group consisting of Ni, Raney Ni, and Pd/C. In some embodiments, the hydrogenation uses a metal catalyst selected from the group consisting of Ni or Raney Ni. In some embodiments, the hydrogenation catalyst is Ni. In some embodiments, the hydrogenation catalyst is Raney Ni.

In some embodiments, the metal reduction uses a metal selected from the group consisting of lithium, sodium, and potassium, and the metal reduction optionally uses a catalyst. In some embodiments the catalyst is naphthalene. In some embodiments, the metal reduction uses a metal that is lithium and a catalyst that is naphthalene.

In some embodiments, the compound of formula (II-C):

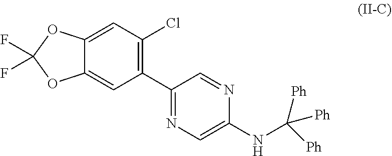 [SMILES: FC1(F)Oc2cc(Cl)c(-c3cnc(NC(c4ccccc4)(c4ccccc4)c4ccccc4)cn3)cc2O1]

is synthesized by coupling a compound of formula (II-D):

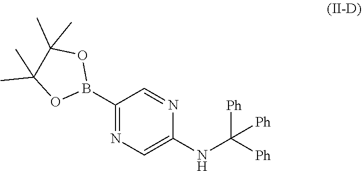 [SMILES: CC1(C)OB(c2cnc(NC(c3ccccc3)(c3ccccc3)c3ccccc3)cn2)OC1(C)C]

and a compound of formula (II-E):

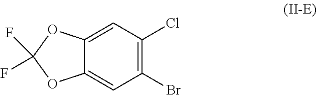 [SMILES: FC1(F)Oc2cc(Cl)c(Br)cc2O1]

in the presence of a coupling catalyst.

In some embodiments, the compound of formula (II-C) is synthesized by coupling a compound of formula (II-D) and a compound of formula(II-E) in the presence of a coupling catalyst, a base, and a polar solvent.

In some embodiments, the coupling catalyst is a palladium-based catalyst. In some embodiments, the palladium-based catalyst is selected from the group consisting of Pd(PPh₃)₄, Pd(OAc)₂, Pd(dppf)Cl₂, Pd(dtbpf)Cl₂, Pd(dba)₂, Pd(PCy₃)₂, Pd(dppe)Cl₂, Pd(t-Bu₃P)₂, PdCl₂[P(o-Tol)₃]₂, benzylbis(triphenylphosphine)palladium(II) chloride, (A-Phos)₂Cl₂Pd, Na₂PdCl₄, SPhos (2-(2′,6″-dimethoxybiphenyl)dicyclohexylphospine), and PdCl₂(PPh₃)₄. In some embodiments, the palladium-based catalyst is selected from the group consisting of Pd(PPh₃)₄, Pd(dppf)Cl₂ and PdCl₂(PPh₃)₄. In some embodiments, the palladium-based catalyst is Pd(PPh₃)₄.

In some embodiments, the base is selected from the group consisting of lithium hydroxide, sodium hydroxide, potassium hydroxide, ammonium hydroxide, potassium acetate, sodium acetate, tripotassium phosphate, sodium butoxide, potassium butoxide, sodium carbonate, potassium carbonate, cesium carbonate, sodium bicarbonate, potassium bicarbonate, calcium hydroxide, and triethylamine. In some embodiments, the base is selected from the group consisting of sodium acetate, potassium acetate, and tripotassium phosphate. In some embodiments, the base is potassium acetate. In some embodiments, the basie is tripotassium phosphate. In some embodiments, the base is cesium fluoride.

In some embodiments, the polar solvent is selected from the group consisting of water, acetic acid, formic acid, methanol, ethanol, n-propanol, t-butanol, DMF, 1,4-dioxane, and combinations thereof. In some embodiments, the polar solvent comprises a combination of at least two of water, DMF, and 1,4-dioxane. In some embodiments, the polar solvent comprises a combination of water and DMF. In some embodiments, the polar solvent comprises a combination of DMF and 1,4-dioxane. In some embodiments, the polar solvent comprises a combination of water and 1,4-dioxane. In some embodiments, the polar solvent is 1,4-dioxane. In some embodiments, the polar solvent is DMF.

In some embodiments, the coupling reaction is conducted at a temperature from more than about 10° C., more than about 20° C., more than about 30° C., more than about 40° C. more than about 50° C., more than about 60° C., more than about 70° C., more than about 80° C., more than about 90° C., more than about 100° C., more than about 110° C., more than about 120° C. more than about 130° C., more than about 140° C., less than about 150T° C. less than about 140° C., less than about 130° C., less than about 120° C. less than about 110° C., less than about 100° C., less than about 90° C., less than about 80° C., less than about 70° C., less than about 60° C., less than about 50° C., less than about 40° C., less than about 30° C., less than about 20° C. from about 10° C. to about 150° C., from about 20C to about 140° C., from about 30° C. to about 130° C., from about 40° C. to about 120° C., from about 50C to about 110° C. from about 60° C. to about 110° C., from about 70° C. to about 100° C., from about 70° C. to about 90° C., from about 80° C. to about 90° C., from about 70° C. to about 80° C., from about 75° C. to about 85° C., or from about 85° C. to about 95° C.

In some embodiments, the compound of formula (II-D):

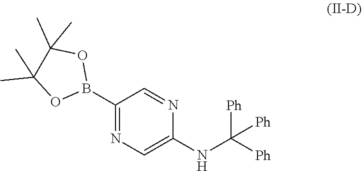 [SMILES: CC1(C)OB(c2cnc(NC(c3ccccc3)(c3ccccc3)c3ccccc3)cn2)OC1(C)C]

is synthesized by treating a compound of formula (II-F)

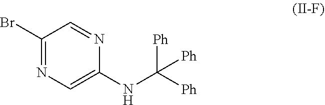 [SMILES: Brc1cnc(NC(c2ccccc2)(c2ccccc2)c2ccccc2)cn1]

with bis(pinacolato)diboron in the presence of a palladium-based catalyst, a base, and a polar solvent.

In some embodiments, the palladium-based catalyst is selected from the group consisting of Pd(PPh₃)₄, Pd(OAc)₂, Pd(dppf)Cl₂, Pd(dtbpf)Cl₂, Pd(dba)₂, Pd(PCy₃)₂, Pd(dppe)Cl₂, Pd(t-Bu₃P)₂, PdCl₂[P(o-Tol)₃]₂, benzylbis(triphenylphosphine)palladium(II) chloride, (A-Phos)₂Cl₂Pd. Na₂PdCl₄, and PdCl₂(PPh₃)₄. In some embodiments, the palladium-based catalyst is selected from the group consisting of Pd(PPh₃)₄, Pd(dppf)Cl₂ and PdCl₂(PPh₃)₄. In some embodiments, the palladium-based catalyst is Pd(dppf)Cl₂.

In some embodiments, the base is selected from the group consisting of lithium hydroxide, sodium hydroxide, potassium hydroxide, ammonium hydroxide, potassium acetate, sodium acetate, tripotassium phosphate, sodium butoxide, potassium butoxide, sodium carbonate, potassium carbonate, cesium carbonate, sodium bicarbonate, potassium bicarbonate, calcium hydroxide, and triethylamine. In some embodiments, the base is selected from the group consisting of sodium acetate, potassium acetate, and tripotassium phosphate. In some embodiments, the base is potassium acetate.

In some embodiments, the polar solvent is selected from the group consisting of water, acetic acid, formic acid, methanol, ethanol, n-propanol, t-butanol, and 1,4-dioxane. In some embodiments, the polar solvent is 1,4-dioxane.

In some embodiments, the compound of formula (II-F):

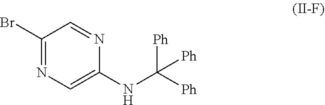 [SMILES: Brc1cnc(NC(c2ccccc2)(c2ccccc2)c2ccccc2)cn1]

is synthesized from 2-amino-5-bromopyrazine.

In some embodiments, the compound of formula (II-F) is synthesized from 2-amino-5-bromopyrazine by treating 2-amino-5-bromopyrazine with triphenylmethylchloride in the presence of a tertiary amine base. In some embodiments, the compound of formula(II-F) is synthesized from 2-amino-5-bromopyrazine by treating 2-amino-5-bromopyrazine with triphenylmethylchloride in the presence of a tertiary amine base in an aprotic polar solvent. In some embodiments, the aprotic polar solvent is dichloromethane. In some embodiments the tertiary amine base is triethylamine or pyridine. In some embodiments, the tertiary amine base is triethylamine. In some embodiments, the tertiary amine basis is pyridine. In some embodiments the tertiary amine base is triethylamine and the aprotic polar solvent is dichloromethane.

In some embodiments, the compound of formula (II-F) is in a form selected from the group consisting of a solid, a liquid, and a solution. In some embodiments, the solid is a crystalline solid or an amorphous solid. In some embodiments, the solid is a crystalline solid.

In some embodiments, the compound of formula (II-B):

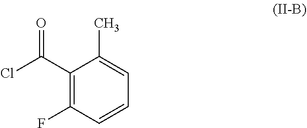

is synthesized by treating a compound of formula (II-G):

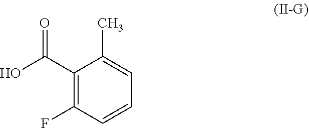

with an acyl halide preparation agent.

In some embodiments, the acyl halide preparation agent is selected from the group consisting of oxalyl chloride, thionyl chloride, phosphoryl chloride, phosphorus trichloride, methanesulfonyl chloride, trichloromethanesulfonyl chloride, tert-butyl hypochlorite, dichloromethyl methyl ether, methoxyacetyl chloride, cyanuric chloride, N-chlorosuccinamide, N-chlorophthalimide, and trimethylsilyl chloride. In some embodiments, the acyl halide preparation agent is selected from the group consisting of oxalyl chloride, thionyl chloride, phosphoryl chloride, and phosphorus trichloride. In some embodiments, the acyl halide preparation agent is oxalyl chloride.

### EXAMPLES

The examples provided herein are for illustrative purposes only and shall not limit the scope of the appended claims. The starting materials and reagents used for the synthesis of the compounds described herein were synthesized or were obtained from commercial sources, such as, but not limited to, Sigma-Aldrich, Acros Organics, Fluka, and Fischer Scientific. The identity, yield, and purity of the products of the larger-scale preparations described herein were verified and determined using standardized analytical methods developed on the basis of reference standards prepared by independent synthesis. In the Examples below, the nomenclature “n-mX”, where “n-m” recites a range of integers, denotes the approximate factor by which a given solute was concentrated by removing excess solvent by selective solvent stripping (solvent vaporization). For example, “the solution was concentrated to 4-5×” means sufficient solvent was removed to increase the concentration of non-volatile solute(s) to from 4 to 5 times the original concentration; if the original concentration was 0.2M, “concentrated to 4-5×” results in a solution where the solute concentrations has increased to from about 0.8M to about 1.0M.

### Example 1—Preparation of Compound 1.3

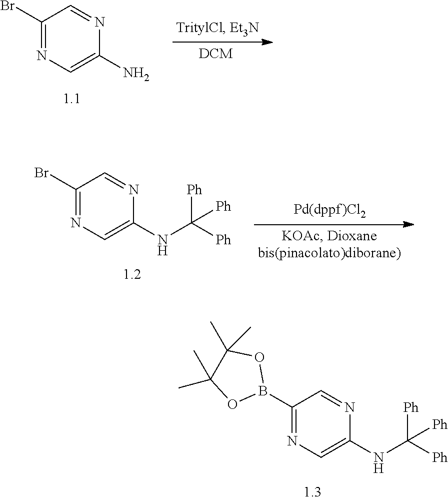 [SMILES: Nc1cnc(Br)cn1 ; Brc1cnc(NC(c2ccccc2)(c2ccccc2)c2ccccc2)cn1 ; CC1(C)OB(c2cnc(NC(c3ccccc3)(c3ccccc3)c3ccccc3)cn2)OC1(C)C]

### Preparation of 5-bromo-N-tritylpyrazin-2-amine (1.2)

5-bromopyrazin-2-amine (1.1)(3.9 kg, 22.41 moles) was added to DCM (23-24 L). At room temperature, the solution was stirred and 3.6 kg triethylamine(Et₃N) (35.57 mol) was added via head tank. The reaction mixture was cooled to 0-10° C., and a solution of triphenylmethyl chloride (7.0 g, 25.11 mol) in DCM (11-13 L) in HDPE drum was slowly added via head tank, not exceeding 0-20° C. The reaction proceeded at 15-20° C. for 5-9 hr. Upon completion, the reaction was quenched with water (˜4 L), and was allowed to stir at 10-25° C. for 40-60 min. The layers were separated, and 5% NaCl_{(aq)} (˜4 L) was added to the organic layer. The mixture was stirred for 40-60 mins, and the layers were separated. The solution was concentrated to 4-5×, methyl tertiary-butyl ether (MTBE) (15-20 kg) was added, and the solution was re-concentrated to 4-5×. This process was repeated 3 times. After the third iteration of this process, the subsequent mixture was stirred at 10-20° C. for 10-20 min. The resulting solid was filtered to produce a wet cake. The cake was dried at 40-50° C. for 16-20 hr. Yield=8.33 kg, purity=96.8%.

### Preparation of 5-(4,4,5,5-tetramethyl-1,3,2-dioxaborolan-2-yl)-N-tritylpyrazin-2-amine (1.3)

1,4-dioxane (28-30 L) was added to 5-bromo-N-tritylpyrazin-2-amine (1.2)(6.8 kg, 16.33 moles), bis(pinacolato)diboron (4.95 kg, 19.5 moles), and potassium acetate (KOAc)(2.4 kg). At room temperature, the solution purged with nitrogen 3 times. Pd(dppf)Cl₂ (1.17 kg, 1.66 moles) was added, and the solution was purged with nitrogen 3 times. The reaction proceeded at 80-90° C. for 16-20 hr. Upon completion, the reaction was cooled to 20-30° C., and the solution was filtered and concentrated to 2×-4×. The solution was immediately taken to the next step without further purification.

### Example 2—Preparation of Compound 2.3

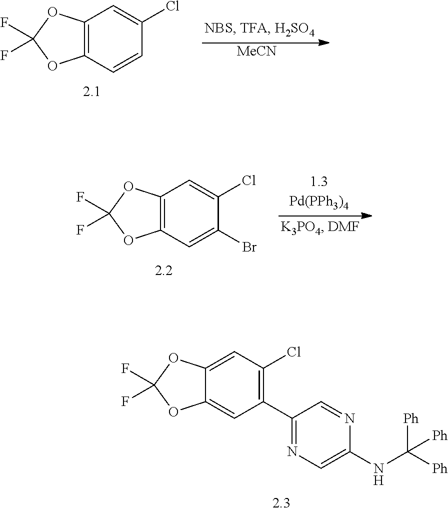 [SMILES: "MeCN" ; FC1(F)Oc2ccc(Cl)cc2O1 ; FC1(F)Oc2cc(Cl)c(Br)cc2O1 ; FC1(F)Oc2cc(Cl)c(-c3cnc(NC(c4ccccc4)(c4ccccc4)c4ccccc4)cn3)cc2O1]

### Preparation of 5-bromo-6-chloro-2,2-difluorobenzo[d][1,3]dioxole (2.2)

5-chloro-2,2-difluorobenzo[d][1,3]dioxole (2.1)(3.0 kg, 15.58 moles) was added to MeCN (˜2.6 L). At 10-20° C., N-bromosuccinamide (NBS) (2.8-3.2 kg, 17-19 moles) was added to the solution. ˜6.5 kg trifluoroacetic acid (TFA) was added slowly at 10-20° C., followed by the slow addition of ˜6.7 kg sulfuric acid (H₂SO₄) at 10-20° C. The reaction proceeded at 15-20° C. for 24-36 hr. A second batch of NBS (˜0.35 kg) was added, and the reaction proceeded at 15-20° C. for 24-36 hr. A third batch of NBS (˜0.35 kg) was added, and the reaction proceeded at 15-20° C. for 24-36 hr. A fourth batch of NBS (˜0.35 kg) was added, and the reaction proceeded at 15-20° C. for 24-36 hr. Upon near completion, water (˜5.8 L) was added, and the solution was cooled to 0-5° C. Methyl tertiary-butyl ether (MTBE) (˜5 kg) was added, and the product was extracted with MTBE 3 times. Keeping the internal temperature at 0-15° C., the organic layer was basified with 10% NaOH_{(aq)} (˜5 kg) to pH˜10-12. The subsequent mixture was stirred at 0-15° C. for 40-60 min. They layers were separated, and the subsequent organic layer was washed with water (˜4 L). Keeping the temperature less than 30° C., the organic layer was concentrated to 2×-6× under reduced pressure. Yield=3.54 Kg, purity=87.8%.

### Preparation of 5-(6-chloro-2,2-difluorobenzo[d][1,3]dioxol-5-yl)-N-tritylpyrazin-2-amine (2.3)

DMF (˜20 L) was added to freshly prepared 5-(4,4,5,5-tetramethyl-1,3,2-dioxaborolan-2-yl)-N-tritylpyrazin-2-amine (1.3), and the solution was stirred for 15-30 min. This solution was added to previously prepared 5-bromo-6-chloro-2,2-difluorobenzo[d][1.3]dioxole (2.2) MTBE solution (2.28 kg, 12.44 moles, net quantity as 1.0×). CsF (˜4 kg) and water (˜0.017 kg) were added, and the solution was degassed 3× with nitrogen. Pd(PPh₃)₄ (0.94 kg, 0.81 moles) was added under nitrogen, degassed 3× with nitrogen, and the reaction proceeded at 80-90° C. for 1-2 hr. Upon completion, the reaction was cooled to 15-25° C., and water (˜4 L) was added. DCM ( ) was added, the mixture was stirred for 30-60 min at 15-25° C., and then the layers were allowed to separate. The organic layer was collected, and the reaction container was cleaned with water and backwashed with DCM 3×. The organic layers were combined and concentrated to 13×-14×. MeOH was added and evaporated three times, producing a solid that was centrifuged. The mother liquor was removed, and the resulting solid was dissolved in DCM (13×-14×). The process of adding and evaporating MeOH was repeated 3 more times, and the mixture was centrifuged. The mother liquor was separated, and the resulting solid was dried at 40-50° C. for 5-10 hr. The solid was dissolved in DCM, stirring for 30-60 mins at 15-25° C. until clear, and the solution was filtered through silica gel (5×-8×) twice. The resulting mother liquor was concentrated to 13×-14×, and dissolved with MeIH (10×-11×), stirring for 30-60 min at 10-20° C. The mixture was cetrifuged, and the mother liquor was separated. This centrifugation process was repeated, and the resulting solid was dried at 40-50° C. for 16-20 hr. Yield=3.64 kg, purity=99%.

### Example 3—Preparation of Compound 3.2

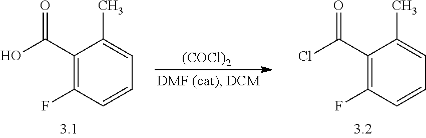

### Preparation of 2-fluoro-6-methylbenzoyl chloride (3.2)

DCM (2.5×-3.0×) and DMF (0.00019×-0.00020×) were added to 2-fluoro-6-methylbenzoic acid (3.1)(0.88 kg, 5.71 moles). At 20-25° C., oxalyl chloride ((COCl)₂)(0.98 kg, 7.72 moles) was added to the solution. The reaction proceeded at 20-30° C. for 16-20 hr. A second batch of DMF (0.00019×-0.00020) was added, and the reaction proceeded at 20-30° C. for 10-12 hr. Toluene (5×-6×kg) was added, and the reaction was concentrated to 2-3×, keeping the internal temperature less than 40° C. This step was repeated, and maintained for immediate use.

### Example 4—Preparation of Compound 4.2

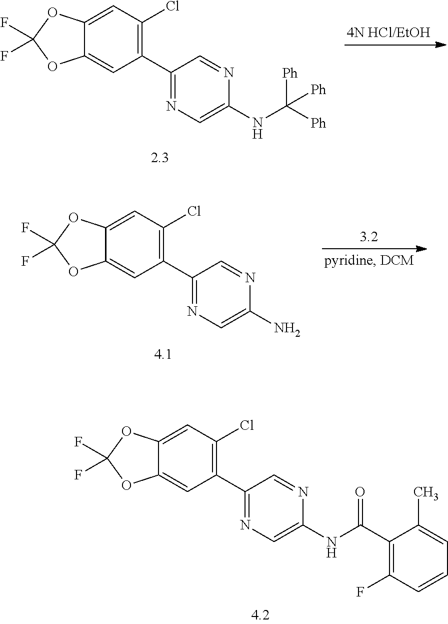 [SMILES: FC1(F)Oc2cc(Cl)c(-c3cnc(NC(c4ccccc4)(c4ccccc4)c4ccccc4)cn3)cc2O1 ; Nc1cnc(-c2cc3c(cc2Cl)OC(F)(F)O3)cn1 ; Cc1cccc(F)c1C(=O)Nc1cnc(-c2cc3c(cc2Cl)OC(F)(F)O3)cn1]

### Preparation of 5-(6-chloro-2,2-difluorobenzo[d][1,3]dioxol-5-yl)pyrazin-2-amine (4.1)

Ethyl alcohol (8.5×-9.5×) was added to 5-(6-chloro-2,2-difluorobenzo[d][1,3]dioxol-5-yl)-N-tritylpyrazin-2-amine (2.3)(3.5 kg, 6.56 moles). At 10-20° C., a solution of 4N HCl in ethyl alcohol (3.0×-3.3×) was added dropwise via head tank at 10-20° C. The reaction proceeded at 10-20° C. for 2-4 hr. The reaction was filtered via Buchner funnel, and the mother liquor was collected and centrifuged. The mother liquor was removed, and the subsequent crude solid was dissolved in 2-MeTHF (5.0×-5.5×), stirring for 15-30 min at 10-20° C. 4% NaHCO_{3(aq)} (5.0×-5.5×) was added dropwise at 10-20° C. via head tank until the solution reached pH 8-9. The mixture was stirred for 20-40 min at 10-20° C., and then rested for 15-30 min. The organic layer was collected, and a backwash of the aqueous with 2-MeTHF was performed twice. The organic layers were combined, and 2-mercaptobenzoic acid (0.09×-1.2×) was added. The mixture was stirred at 10-20° C. for 1-2 hr. At 10-20° C., 5% Na₂CO_{3(aq)} (3.9×-4.1×) was added, stirred for 15-30 mins, and rested for 15-30 mins. The organic layer was collected, and the Na₂CO₃ wash was repeated 3 more times. This was followed by three additional washes with brine (4.9×-5.1×). The organic layer was collected, and n-heptane (5.0×-6.5×) was added dropwise via headtank at 10-20° C. The solution was cooled to 0-5° C. and stirred for 2-3 hr. The resulting solid was filtered, and the product was dried at 40-50° C. for 16-20 hr. Yield=1.68 Kg, purity=99.2%.

### Preparation of N-(5-(6-chloro-2,2-difluorobenzo[d][1,3]dioxol-5-yl)pyrazin-2-yl)-2-fluoro-6-methylbenzamide (4.2)

5-(6-chloro-2,2-difluorobenzo[d][1,3]dioxol-5-yl)pyrazin-2-amine (4.1)(1.09 kg, 3.82 moles) and DCM (13.5×-14.5×) were added to freshly prepared 2-fluoro-6-methylbenzoyl chloride (3.2). Under nitrogen atmosphere, the reaction was cooled to 0-5° C., and pyridine (0.98 kg, 12.4 moles) was slowly added via head tank below 5° C. The reaction proceeded at 0-5° C. for 16-20 hr. The addition of pyridine (0.98 kg, 12.4 moles) was performed until the reaction completed. Upon completion, 5% NaHCO_{3(aq)} (10×-10.5×) was added dropwise via head tank, keeping the temperature below 20° C. The mixture was stirred at 10-20° C. for 1-2 h, and then allowed to rest for 15-30 min. The bottom layer was collected, and DCM (6.5×-7.0×) was added. The solution was stirred for 15-30 min at 10-20° C., and then allowed to rest for 15-30 mins. The organic layer was collected and concentrated to 5-6×, keeping the inner temperature below 40° C. Three times, THF (10×-11×) was added and concentrated to 5-6×, keeping the inner temperature less than 40° C. MeOH (4×-5×) was added at 10-20° C., and 2M NaOH,) (6×-7×) was added at 20-30° C. The reaction was stirred for 3-6 hr at 30-40° C., or until completion. The reaction was cooled to 5-10° C. 6M HCl (2-5×) was added dropwise via headtank at 20° C., adjusting to pH=9-10. The solution was concentrated to 15×-16×, keeping the inner temperature below 40° C. Water (5×-6×) and ethyl acetate (10×-10.5×) were added: the mixture was stirred for 15-30 min at 30-40° C., and then allowed to rest for 15-30 min at 30-40° C. The organic layer was collected, and this process was repeated. The organic layers were combined and washed with water (10×-10.5×) three times. The organic layer was concentrated to 5×-6×, keeping the inner temperature below 40° C. n-heptane (10×-10.5×) was added, the solution was cooled to 10-15° C., and the material was filtered and washed with n-heptane (1×-2×). The subsequent solid was dissolved in MeOH (19×-21×) at 40-50° C., stirring for 1-2 hr. The solution was cooled to 20-25° C., and the resulting solid was filtered and rinsed with MeOH (1×-2×). The resulting solid was mixed with 0.1× silicathiol, stirred for 1-2 hr at 20-30° C., filtered, and rinsed with MeOH (1×-2×). Concentrate to 5-6×, keeping an inner temperature below 50° C., and then add isopropyl alcohol (10×-10.5×). Concentrate to 5-6×, keeping an inner temperature below 50° C. and then heat to 80-90° C. The solution was stirred for 1-2 hr at 80-90° C., and then cooled to −10-0° C. The mixture was stirred for 1-2 hr at −10-0° C., and the resulting solid was filtered and rinsed with cold isopropanol (1×-2×). The wet cake (1.24 kg) was dissolved in ethyl acetate (6×-7×), stirring for 1-2 hr at 20-30° C. The solution was concentrated to 3×-4×, keeping the inner temperature below 40° C. Isopropyl alcohol (9.5×-10.5×) was added, and the solution was concentrated to 4-5×, keeping an inner temperature below 40° C. Cool the solution to −10-0° C., filter the resulting solid, and rinse with cold isopropyl alcohol. Dry product at 40-50° C. for 16-20 hr. Yield=1.09 Kg, purity=100%.

While certain embodiments of the present invention have been shown and described herein, it will be obvious to those skilled in the art that such embodiments are provided by way of example only. Numerous variations, changes, and substitutions will now occur to those s killed in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A process for the synthesis of compounds of Formula (I): 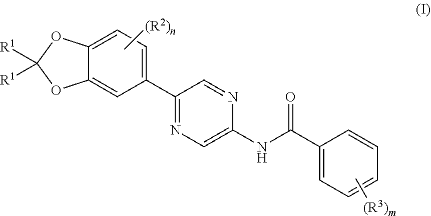
or pharmaceutically acceptable salts thereof, wherein:
R¹ is independently selected at each occurrence from hydrogen, halogen and C₁-C₃ alkyl optionally substituted with one or more substituents independently selected at each occurrence from halogen, —OR′, —CN, —N(R′)₂ and —NO₂;
R² and R³ are independently selected at each occurrence from halogen and C₁-C₃ alkyl optionally substituted with one or more substituents independently selected at each occurrence from halogen, —OR′, —CN, —N(R′)₂ and —NO₂;
or, when both R¹ are independently C₁-C₃ alkyl, the two R¹ groups are taken together with the atom to which they are attached to form a carbocycle;
n is 0, 1, 2 or 3;
m is 0, 1, 2, 3, 4, or 5; and
R′ is independently selected at each occurrence from hydrogen; and C₁₋₆ alkyl, C₂₋₆ alkenyl, and C₂₋₆ alkynyl, each optionally substituted with one or more substituents independently selected at each occurrence from halogen, —CN, —NO₂, —OH, —NH₂, and OCH₃;
wherein the process comprises contacting a compound of Formula (I-A) 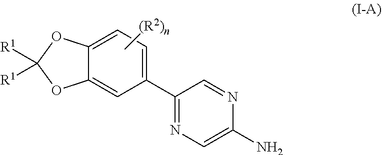
with a compound of Formula (I-B) 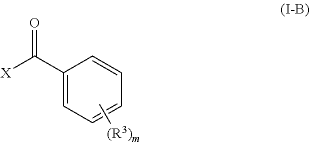
in the presence of a tertiary amine base and an aprotic polar solvent, wherein X is —Cl, —Br, —I, —CN, —N₃, —OCH₃, —OCH₂CH₃, —OC₆H₅, —OC₆H₄-4-NO₂, —OC(O)CH₃, —OC(O)C₆H₅, —O(SO₂)CH₃, or —O(SO₂)C₆H₄-4-CH₃.

2. The process of claim 1, wherein the tertiary amine base is selected from the group consisting of pyridine, triethylamine, triisopropyl amine, 2-tert-butyl-1,1,3,3-tetramethylguanidine, 4-dimethylaminopyridine, N,N-diisopropylethylamine and N-methylmorpholine.

3. The process of claim 1, wherein the aprotic polar solvent is selected from the group consisting of chloroform, dichloromethane, and mixtures thereof.

4. The process of claim 1, wherein the compound of Formula (I-A) 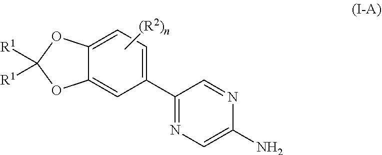
is synthesized by treating a compound of formula (I-C) 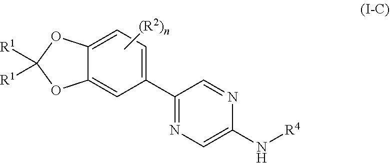
with an acid, wherein R⁴ is selected from the group consisting of trityl, t-butyl, t-butoxycarbonyl, p-tolyl, benzoyl, acetyl and benzyl.

5. The process of claim 4, wherein the acid is selected from the group consisting of trifluoroacetic acid, sulfuric acid and hydrochloric acid.

6. The process of claim 1, wherein the compound of Formula (I-A) 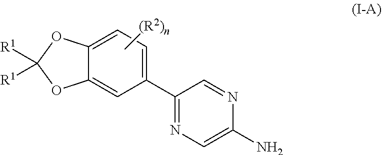
is synthesized by subjecting a compound of formula (I-C) 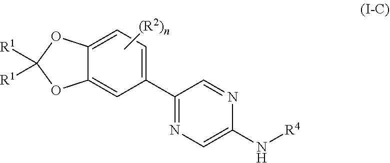
to a hydrogenation, wherein R⁴ is selected from the group consisting of trityl, t-butyl, p-tolyl, and benzyl.

7. The process of claim 4, wherein the compound of formula (I-C) 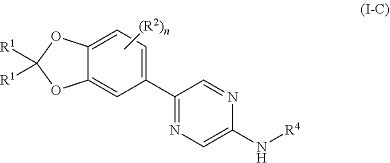
is synthesized by coupling a compound of formula (I-D) 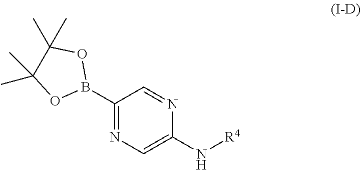
and a compound of formula (I-E) 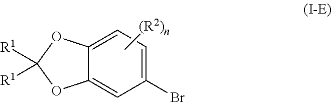
in the presence of a coupling catalyst.

8. The process of claim 7, wherein the coupling catalyst is a palladium-based catalyst.

9. The process of claim 8, wherein the palladium-based catalyst is selected from the group consisting of Pd(PPh₃)₄, Pd(dppf)Cl₂ and PdCl₂(PPh₃)₄.

10. The process of claim 7, wherein the coupling is conducted at a temperature from about 80° C. to about 90° C.

11. The process of claim 7, wherein the compound of formula (I-D) 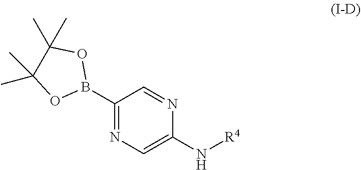
is synthesized by treating a compound of formula (I-F) 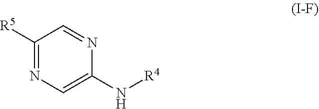
with bis(pinacolato)diboron in the presence of a second palladium-based catalyst, a base and a polar solvent,
wherein R⁵ is independently selected from a halogen, —O(SO₂)CH₄-4-CH₃, and —O(SO₂)CH₃.

12. The process of claim 11, wherein the second palladium-based catalyst is Pd(dppf)Cl₂.

13. The process of claim 11, wherein the base is potassium acetate.

14. The process of claim 11, wherein the compound of formula (I-F) is 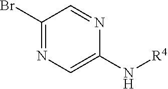
and is synthesized from 2-amino-5-bromopyrazine.

15. The process of claim 14, wherein the compound of formula (I-F) is a crystalline solid.

16. The process of claim 1, wherein the compound of formula (I-B) 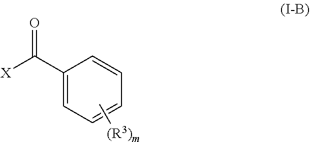
is synthesized by treating a compound of formula (I-G) 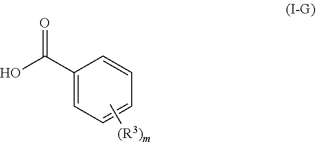
with an acyl halide preparation agent.

17. The process of claim 16, wherein the acyl halide preparation agent is selected from the group consisting of oxalyl chloride, thionyl chloride, phosphoryl chloride, and phosphorus trichloride.
